(19) United States
(12) Patent Application Publication
(10) Pub. No.: US 2019/0354176 A1
(43) Pub. Date: Nov. 21, 2019

(54) INFORMATION PROCESSING APPARATUS, INFORMATION PROCESSING METHOD, AND COMPUTER READABLE RECORDING MEDIUM

(71) Applicant: OLYMPUS CORPORATION, Tokyo (JP)

(72) Inventors: Kazuhito HORIUCHI, Tokyo (JP); Nobuyuki WATANABE, Yokohama-shi (JP); Yoshioki KANEKO, Tokyo (JP); Hidetoshi NISHIMURA, Tokyo (JP)

(73) Assignee: OLYMPUS CORPORATION, Tokyo (JP)

(21) Appl. No.: 16/411,840

(22) Filed: May 14, 2019

(30) Foreign Application Priority Data
May 17, 2018 (JP) 2018-095449

(51) Int. Cl. G06F 3/01; G06F 3/16; G10L 15/22; G10L 15/08; G02B 21/36; G02B 21/02; G02B 25/00; G02B 27/00; A61B 1/05; A61B 1/00

(52) U.S. Cl. CPC G06F 3/013; G06F 3/167; G10L 15/22; G10L 15/08; G02B 21/367; G10L 2015/088; G02B 25/001; G02B 27/0093; A61B 1/05; A61B 1/00039; A61B 1/00006; G02B 21/025

(57) ABSTRACT

An information processing apparatus includes a processor comprising hardware, the processor being configured to execute: setting an utterance period, in which an uttering voice includes a keyword having an importance degree of a predetermined value or more, as an important period with respect to user's voice data input from an external device; and allocating a corresponding gaze period corresponding to the set important period to gaze data that is input from an external device and is correlated with the same time axis as in the voice data, and recording the corresponding gaze period in a memory.

This application is based upon and claims the benefit of priority from Japanese Patent Application No. 2018-095449, filed on May 17, 2018, the entire contents of which are incorporated herein by reference.

### BACKGROUND

The present disclosure relates to an information processing apparatus, an information processing method, and a computer readable recording medium.

Recently, in an information processing apparatus that processes information such as image data, there is known a technology in which an attention information is determined with using gaze on the display and voice detection. In the technology, an area having the longest gaze period is extracted, as the attention information, from a plurality of areas of the display, within a predetermined period going back from the time when utterance is detected, and the attention information and voice are recorded with association (refer to JP 4282343 B).

In addition, there is a known a technology in an annotation system, with using an anchor on the display and gaze detection and voice record. On an image displayed by a display device of a computing device, an annotation anchor is displayed at a site closer to a gaze point which is detected by a gaze tracking device and which a user gazes, and information is input to the annotation anchor with voice (refer to JP 2016-181245 A).

### SUMMARY

According to one aspect of the present disclosure, there is proceeded an information processing apparatus including a processor comprising hardware, the processor being configured to execute: setting an utterance period, in which an uttering voice includes a keyword having an importance degree of a predetermined value or more, as an important period with respect to user's voice data input from an external device; and allocating a corresponding gaze period corresponding to the set important period to gaze data that is input from an external device and is correlated with the same time axis as in the voice data, and recording the corresponding gaze period in a memory.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a functional configuration of an information processing system according to a first embodiment;

FIG. 2 is a flowchart illustrating an outline of processing that is executed by an information processing apparatus according to the first embodiment;

FIG. 3 is a view schematically describing a setting method of setting an important period with respect to voice data by a setting unit according to the first embodiment;

FIG. 4 is a view schematically describing a setting method in which an analysis unit according to the first embodiment sets the degree of importance to gaze data;

FIG. 5 is a view schematically illustrating an example of an image that is displayed by a display unit according to the first embodiment;

FIG. 6 is a view schematically illustrating another example of the image that is displayed by the display unit according to the first embodiment;

FIG. 7 is a block diagram illustrating a functional configuration of an information processing system according to a second embodiment;

FIG. 8A is a flowchart illustrating an outline of processing that is executed by an information processing apparatus according to the second embodiment;

FIG. 8B is a view schematically describing a setting method in which an analysis unit according to the second embodiment sets the degree of importance to gaze data;

FIG. 9 is a schematic view illustrating a configuration of an information processing apparatus according to a third embodiment;

FIG. 10 is a schematic view illustrating the configuration of the information processing apparatus according to the third embodiment;

FIG. 11 is a block diagram illustrating a functional configuration of the information processing apparatus according to the third embodiment;

FIG. 12 is a flowchart illustrating an outline of processing that is executed by the information processing apparatus according to the third embodiment;

FIG. 13 is a view illustrating an example of gaze mapping image that is displayed by a display unit according to the third embodiment;

FIG. 14 is a view illustrating another example of the gaze mapping image that is displayed by the display unit according to the third embodiment;

FIG. 15 is a schematic view illustrating a configuration of a microscopic system according to a fourth embodiment;

FIG. 16 is a block diagram illustrating a functional configuration of the microscopic system according to the fourth embodiment;

FIG. 17 is a flowchart illustrating an outline of processing that is executed by the microscopic system according to the fourth embodiment;

FIG. 18 is a schematic view illustrating a configuration of an endoscopic system according to a fifth embodiment;

FIG. 19 is a block diagram illustrating a functional configuration of the endoscopic system according to the fifth embodiment;

FIG. 20 is a flowchart illustrating an outline of processing that is executed by the endoscopic system according to the fifth embodiment;

FIG. 21 is a view schematically illustrating an example of a plurality of images corresponding to a plurality of pieces of image data which are recorded by an image data recording unit according to the fifth embodiment;

FIG. 22 is a view illustrating an example of an integrated image corresponding to integrated image data that is generated by an image processing unit according to the fifth embodiment;

FIG. 23 is a block diagram illustrating a functional configuration of an information processing system according to a sixth embodiment; and

FIG. 24 is a flowchart illustrating an outline of processing that is executed by the information processing system according to the sixth embodiment.

### DETAILED DESCRIPTION

Hereinafter, modes for carrying out the present disclosure will be described in detail with reference to the accompanying drawings. Note that, the present disclosure is not limited by the following embodiments. In addition, respective drawings which are referenced in the following description schematically illustrate a shape, a size, and a positional relationship to a certain extent capable of understanding the content of the present disclosure. That is, the present disclosure is not limited to shapes, sizes, and positional relationships which are exemplified in the respective drawings.

### First Embodiment

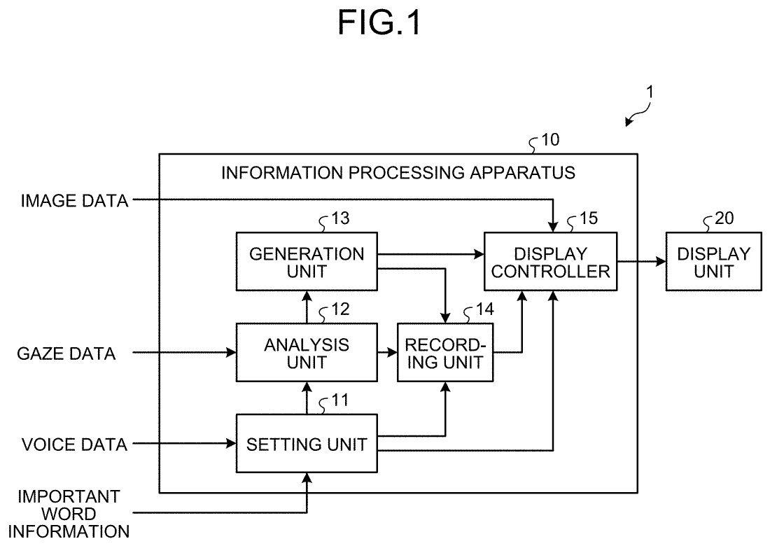

Configuration of Information Processing System FIG. 1 is a block diagram illustrating a functional configuration of an information processing system according to a first embodiment. An information processing system 1 illustrated in FIG. 1 includes an information processing apparatus 10 that performs various kinds of processing with respect to gaze data, voice data, and image data which are input from an outer side, and a display unit 20 that displays various pieces of data which are output from the information processing apparatus 10. Note that, the information processing apparatus 10 and the display unit 20 are connected to each other in a wireless or wired manner.

Configuration of Information Processing Apparatus

First, a configuration of the information processing apparatus 10 will be described.

The information processing apparatus 10 illustrated in FIG. 1 is executed by using a processing device for example, a server, a PC, a ASIC, a FPGA or the like, in which implemented a program, and various pieces of data are input to the information processing apparatus 10 through a network, or various pieces of data which are acquired by an external device are input thereto. As illustrated in FIG. 1, the information processing apparatus 10 includes a setting unit 11, an analysis unit 12, a generation unit 13, a recording unit 14, and a display controller 15.

The setting unit 11 sets an important period of user's voice data that is input from an outer side. Specifically, the setting unit 11 sets an important period of user's voice data that is input from an outer side based on important word information that is input from an outer side. The user's voice data that is input from an outer side is generated by a voice input unit such as a microphone (not illustrated). For example, in a case where a keyword input from an outer side represents “cancer”, “bleeding”, and the like, and the corresponding importance index is “10” and “8” to each, the setting unit 11 sets a period (section or time) in which the keyword occurs to the important period by using known voice pattern matching or the like. Note that, the setting unit 11 may set the important period to include time before and after the period in which the keyword occurs, for example, approximately one second or two seconds. Note that, as the important word information, information that is stored in a database (voice data, textual information) in advance may be used, or may be information that is input by a user (voice data/keyboard input).

With respect to user's gaze data that is input from an outer side and is correlated with the same time axis as in the voice data, the analysis unit 12 allocates a corresponding gaze period (for example, in the case of “cancer”, an index “10”),corresponding to the important period of the voice data which is set by the setting unit 11 and records the corresponding gaze period in the recording unit 14. Here, with regard to the corresponding gaze period, a rank is set in correspondence with an index of a keyword in a gaze period of a gaze of a user in the important period in which the important keyword occurs in the voice data. In addition, the analysis unit 12 analyzes the degree of attention of the gaze of the user based on the gaze data, which is input from an outer side, for predetermined time for which the gaze of the user is detected. Here, the gaze data is based on a cornea reflection method. Specifically, the gaze data is data that is generated by imaging a pupil point on the cornea and a reflection point by an optical sensor that is a gaze detection unit when near infrared rays are emitted to the cornea of a user from an LED light source that is provided in a gaze detection unit (eye tracking device) (not illustrated hear). In addition, the gaze data is obtained by calculating a gaze of the user from a pattern of the pupil point of the user and the reflection point which is based on an analysis by image processing or the like with respect to data that is generated when the optical sensor captures images of the pupil point on the cornea and the reflection point.

In addition, although not illustrated in the drawing, at the time measuring the gaze data with a device incorporating the gaze detection unit, the corresponding image data is presented to the user, and then measure the gaze data. In a case where a use aspect is an endoscopic system or an optical microscope, a field of view that is presented to detect a gaze becomes a field of view of image data, and thus a relative positional relationship of an observation field of view with respect to absolute coordinates of an image does not vary. In addition, in the use aspect of the endoscopic system or the optical microscope, when performing recording as a moving image, gaze detection data and an image that is recorded or presented simultaneously with detection of the gaze are used to generate mapping data of the field of view.

On the other hand, in a use aspect of a whole slide imaging (WSI), a user observes a part of a whole slide image as a field of view, and thus the relative position of the observation field of view to the whole image varies with the passage of time. In this case, information indicating which portion of the image data is presented as the field of view, that is, time information of switching of absolute coordinates of a display area is also recorded in synchronization with information of the gaze and voice.

The analysis unit 12 analyzes the degree of attention of a gaze (gaze point) by detecting any one of a movement speed of the gaze, a movement distance of the gaze in a constant time, and a residence time of the gaze in a certain area, based on the user's gaze data which is input from an outer side for a predetermined time. Note that, the gaze detection unit (not illustrated) may be placed at a predetermined location and may image a user to detect the gaze, or may be worn on the user and may image the user to detect the gaze. In addition, the gaze data may be generated through pattern matching that is known in addition to the above-described configurations.

The generation unit 13 generates gaze mapping data correspond to the input image data from the outer side. The corresponding gaze period analyzed by the analysis unit 12. The generation unit 13 outputs the mapped gaze data to the recording unit 14 and the display controller 15. In this case, when obtaining the gaze mapping data as absolute coordinates of an image as described above, the generation unit 13 uses a relative positional relationship of the absolute coordinates of the image and display area (field of view) of the gaze measurement. For a case where an observation field of view varies every moment, the generation unit 13 gets a variation of absolute coordinates (for example, an upper-left side of a display image is located at which position of original image data in terms of the absolute coordinates) of a display area (a field of view) with the passage of time. Specifically, the gaze position mapping data is generated in which the gaze position corresponding to the gaze period analyzed by the analysis unit 12 is associated with coordinate information of certain area on the image. In addition, the generation unit 13 correlates a trajectory of the user's gaze analyzed by the analysis unit 12 with the image corresponding to the image data that is input from an outer side to generate the gaze mapping data.

The recording unit 14 records, the voice data that is set by the setting unit 11, the gaze data, and the corresponding gaze period analyzed by the analysis unit 12 in correlation with each other, the gaze data and the degree of attention which are analyzed by the analysis unit 12 in correlation with each other the gaze mapping data that is generated by the generation unit 13. The recording unit 14 is constituted by using a volatile memory, a nonvolatile memory, a recording medium, or the like.

The display controller 15 superimposes the gaze mapping data generated by the generation unit 13 on an image corresponding to input image data from an outer side, and outputs the resultant image to the display unit 20 on an outer side to be displayed thereon. The display controller 15 is constituted by using a CPU, an FPGA, a GPU, or the like.

Configuration of Display Unit

Next, a configuration of the display unit 20 will be described.

The display unit 20 displays an image that is input from the display controller 15 and corresponds to the image data or gaze mapping information corresponding to the gaze mapping data. For example, the display unit 20 is constituted by using a display monitor of organic electroluminescence (EL), liquid crystal, or the like.

Processing of Information Processing Apparatus

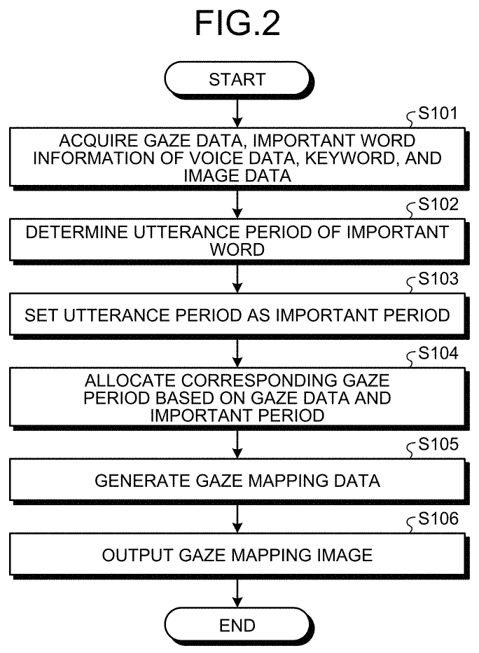

Next, processing of the information processing apparatus 10 will be described. FIG. 2 is a flowchart illustrating an outline of processing that is executed by the information processing apparatus 10.

As illustrated in FIG. 2, first, the information processing apparatus 10 acquires gaze data, voice data, a keyword, and image data which are input from an outer side (Step S101).

Next, the setting unit 11 determines an utterance period in which a keyword that is an important word in the voice data occurs based on the keyword that is input from an outer side (Step S102), and sets the utterance period in which the important word in the voice data occurs as an important period (Step S103). After Step S103, the information processing apparatus 10 transitions to Step S104 to be described later.

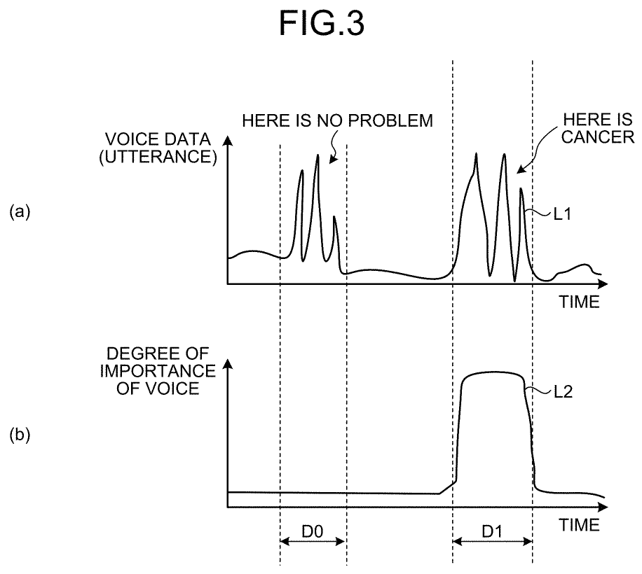

FIG. 3 is a view schematically describing a setting method of setting the important period with respect to the voice data by the setting unit 11. In (a) of FIG. 3 and (b) of FIG. 3, the horizontal axis represents time, the vertical axis in (a) of FIG. 3 represents voice data (utterance), and the vertical axis in (b) of FIG. 3 represents the degree of importance of voice. In addition, a curved line L1 in (a) of FIG. 3 represents a variation of the voice data with the passage of time, and a curved line L2 in (b) of FIG. 3 represents a variation of the degree of importance of voice with the passage of time.

As illustrated in FIG. 3, the setting unit 11 uses voice pattern matching that is known with respect to the voice data, and in a case where a keyword of important words input from an outer side is “cancer”, a period before and after an utterance period (utterance time) of the voice data in which the “cancer” occurs is set as an important period D1 in which the degree of importance is highest. In contrast, the setting unit 11 does not set a period DO, in which a user utters voice but the keyword of the important words is not included, as the important period. Note that, in addition to the know voice pattern matching, after converting the voice data into textual information, with regard to the textual information, the setting unit 11 may set a period corresponding to the keyword as the important period in which the degree of importance is highest.

Returning to FIG. 2, description of processing subsequent to Step S104 will continue.

In Step S104, with respect to gaze data that is user's gaze data input from an outer side, and is correlated with the same time axis as in the voice data, the analysis unit 12 allocates a corresponding gaze period corresponding to an index (for example, in the case of “cancer”, the index is “10”) allocated to the keyword of the important words to a period (time) corresponding to the important period of the voice data which is set by the setting unit 11 to synchronize the voice data and the gaze data, and records the voice data and the gaze data in the recording unit 14. After Step S104, the information processing apparatus 10 transitions to Step S105 to be described later.

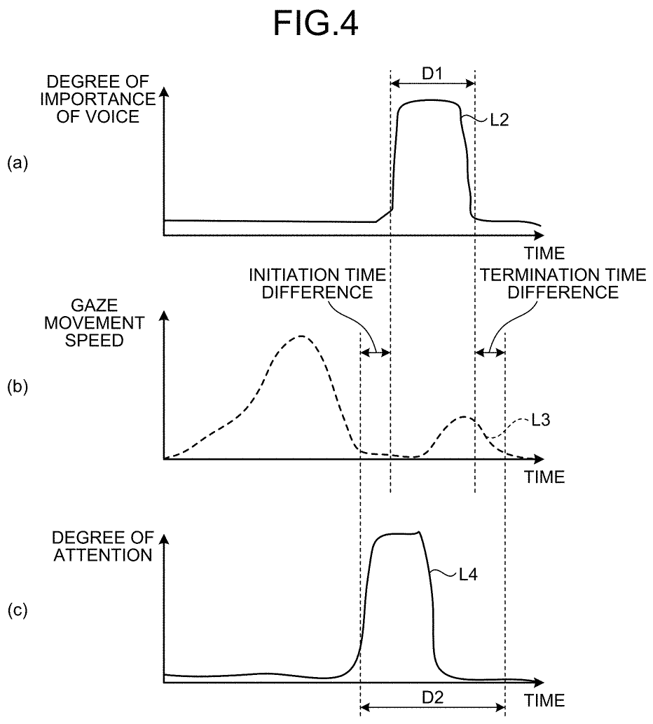

FIG. 4 is a view schematically describing a method of allocating the corresponding gaze period by the analysis unit 12. In (a) of FIG. 4, in (b) of FIG. 4, and (c) of FIG. 4, the horizontal axis represents time, the vertical axis in (a) of FIG. 4 represents the degree of importance of voice, the vertical axis in (b) of FIG. 4 represents a gaze movement speed, and the vertical axis in (c) of FIG. 4 represents the degree of attention.

The analysis unit 12 sets a period of corresponding gaze data based on the period D1 in which the degree of importance of voice is set by the setting unit 11. The analysis unit 12 sets an initiation time difference and a termination time difference with respect to the period D1, and sets corresponding gaze period D2.

Note that, in the first embodiment, calibration processing of calculating a time difference between the degree of attention and pronunciation (utterance) of a user (calibration data) in advance, and of correcting a deviation between the degree of attention and the pronunciation (utterance) of the user based on the calculation result may be performed. Simply, a period in which a keyword of which the degree of importance of voice is high is uttered may be set as the important period, and a period before and after the important period by a constant time or a period shifted from the important period may be set as the corresponding gaze period.

Returning to FIG. 2, description of processing subsequent to Step S105 will continue.

In Step S105, the generation unit 13 generates gaze mapping data in which the corresponding gaze period analyzed by the analysis unit 12 is correlated with an image corresponding to image data.

Next, the display controller 15 superimposes the gaze mapping data generated by the generation unit 13 on the image corresponding to the image data, and outputs the resultant image to the display unit 20 on an outer side (Step S106). After Step S106, the information processing apparatus 10 terminates the processing.

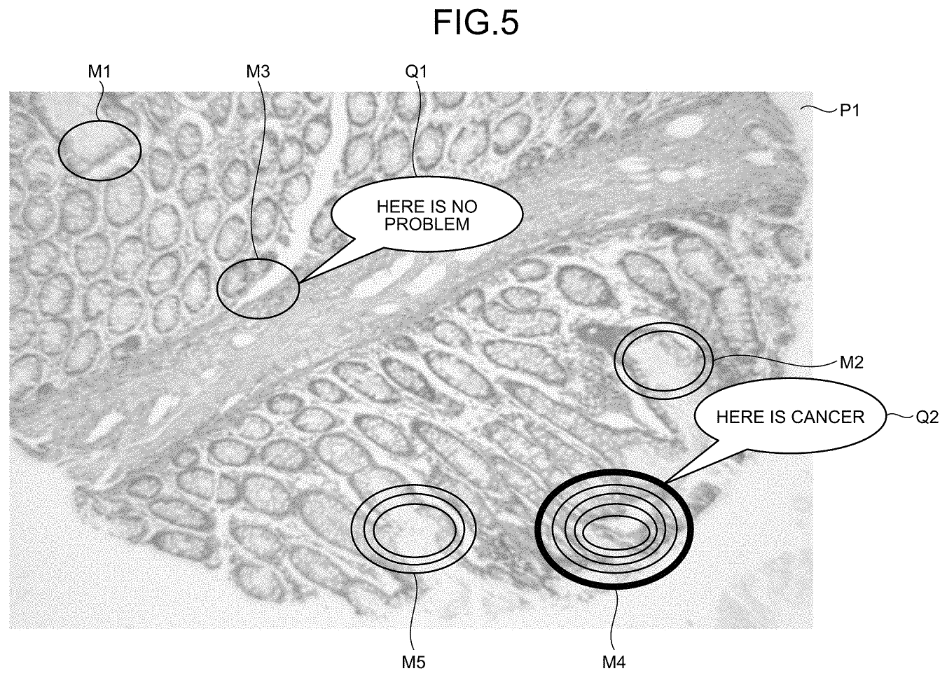

FIG. 5 is a view is a view schematically illustrating an example of an image that is displayed by the display unit 20. As illustrated in FIG. 5, the display controller 15 causes the display unit 20 to display a gaze mapping image P1 in which the gaze mapping data generated by the generation unit 13 is superimposed on an image corresponding to image data. In FIG. 5, the higher the degree of gaze is, the greater the number of contour lines is. The gaze mapping image P1 of heat maps M1 to M5 are displayed on the display unit 20. Here, highlighting display is performed with respect to an area in which a gaze corresponding to a period of which the degree of importance of voice is high is mapped (here, an outer frame of the contour line is made to be bold). Note that, in FIG. 5, the display controller 15 causes the display unit 20 to display the gaze mapping image P1 in a state in which a message Q1 and a message Q2 are superimposed on the gaze mapping image P1 so as to schematically illustrate the content of the degree of importance of voice, but the message Q1 and the message Q2 may not displayed.

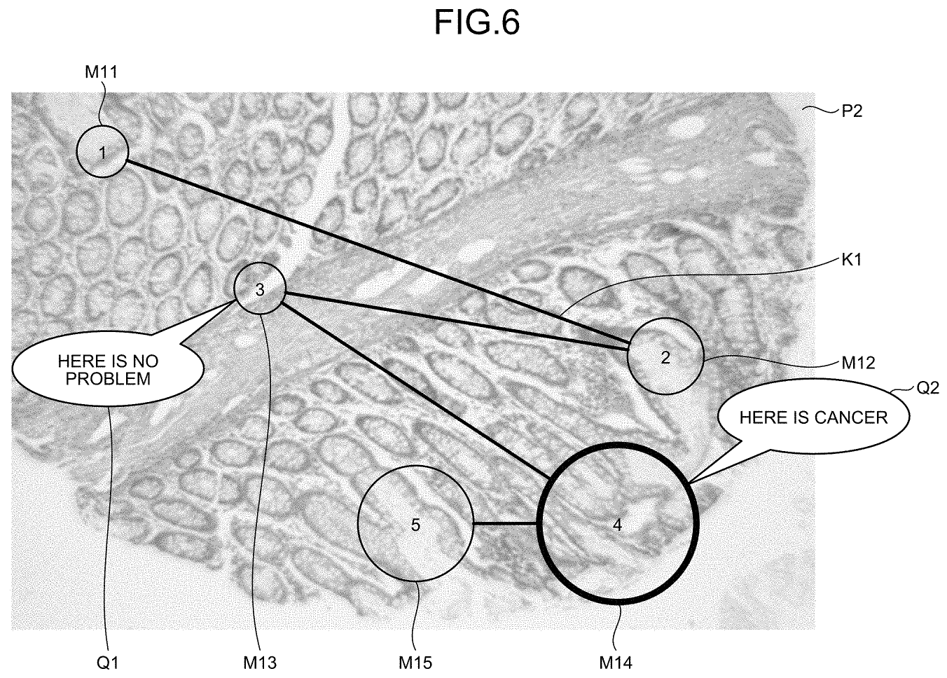

FIG. 6 is a view schematically illustrating another example of an image that is displayed by the display unit 20. As illustrated in FIG. 6, the display controller 15 causes the display unit 20 to display a gaze mapping image P2 in which the gaze mapping data generated by the generation unit 13 is superimposed on an image corresponding to image data. In FIG. 6, the longer a residence time of a gaze is, the greater circular areas of records M11 to M15 are. Here, highlighting display is performed with respect to an area in which a gaze corresponding to a period of which the degree of importance of voice is high is mapped. In addition, the display controller 15 causes the display unit 20 to display a trajectory K1 of a user's gaze and the order of a corresponding gaze period with numbers. Note that, in FIG. 6, the display controller 15 may cause the display unit 20 to display textual information (for example, the message Q1 and the message Q2) obtained by converting voice data that is uttered by a user in a period (time) of each corresponding gaze period by using a known character conversion technology in the vicinity of records M11 to M15, or in a state of being superimposed on the records.

According to the above-described first embodiment, with respect to the gaze data that is correlated with the same time axis as in the voice data, the analysis unit 12 allocates the corresponding gaze period corresponding to an index allocated to the keyword of the important words to a period corresponding to the important period of the voice data which is set by the setting unit 11 to synchronize the voice data and the gaze data, and records the voice data and the gaze data in the recording unit 14. Accordingly, it is possible to understand which period of the gaze data is important.

In addition, in the first embodiment, the generation unit 13 generates the gaze mapping data in which the corresponding gaze period analyzed by the analysis unit 12 and coordinate information of the corresponding gaze period are correlated with an image corresponding to image data that is input from an outer side, and thus a user can intuitively understand an important position on the image.

### Second Embodiment

Next, a second embodiment will be described. In the first embodiment, with respect to the gaze data that is correlated with the same time axis as in the voice data, the analysis unit 12 allocates the corresponding gaze period to a period corresponding to the important period of the voice data which is set by the setting unit 11 to synchronize the voice data and the gaze data, and records the voice data and the gaze data in the recording unit 14. However, in the second embodiment, the corresponding gaze period is allocated to the gaze data based on the degree of attention of a gaze which is analyzed by the analysis unit 12 and the important period that is set by the setting unit 11. In the following description, processing that is executed by an information processing apparatus according to the second embodiment will be described after describing a configuration of an information processing system according to the second embodiment. Note that, the same reference numeral will be given to the same configuration as in the information processing system according to the first embodiment, and detailed description thereof will be omitted.

Configuration of Information Processing System

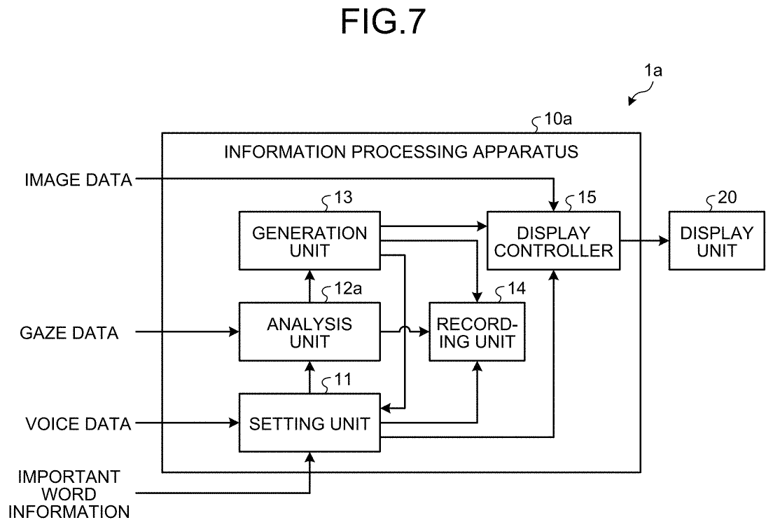

FIG. 7 is a block diagram illustrating a functional configuration of the information processing system according to the second embodiment. An information processing system la illustrated in FIG. 7 includes an information processing apparatus 10a in substitution for the information processing apparatus 10 according to the first embodiment. The information processing apparatus 10a includes an analysis unit 12a in substitution for the analysis unit 12 according to the first embodiment.

The analysis unit 12a analyzes the degree of attention of a gaze (gaze point) by detecting any one of a movement speed of the gaze, a movement distance of the gaze in a constant time, and a residence time of the gaze in a constant area based on gaze data that is user's gaze data input from an outer side and is correlated with the same time axis as in the voice data. In addition, the analysis unit 12a extracts a gaze period for which the degree of attention of the user's gaze is analyzed, allocates the corresponding gaze period to the gaze period of the gaze data before and after the important period of the voice data based on the gaze period and the important period of the voice data which is set by the setting unit 11, and records corresponding gaze period in the recording unit 14.

Processing of Information Processing Apparatus

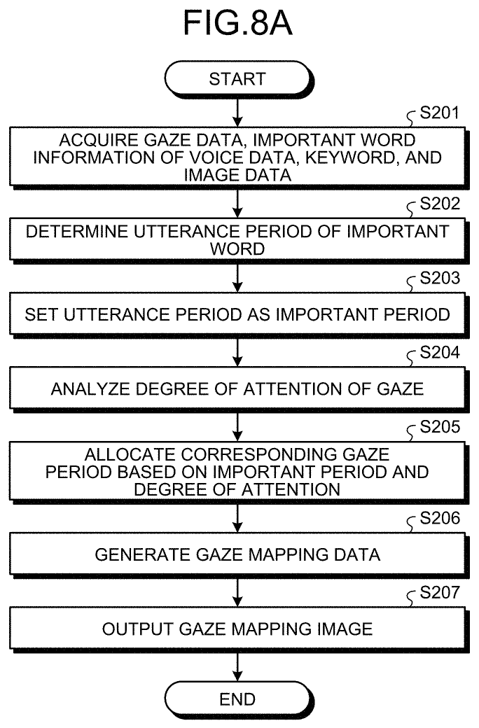

Next, processing that is executed by the information processing apparatus 10a will be described. FIG. 8A is a flowchart illustrating an overview of processing that is executed by the information processing apparatus 10a. In FIG. 8A, Step S201 to Step S203 respectively correspond to Step S101 to Step S103 in FIG. 2.

In Step S204, the analysis unit 12a detects a movement speed of a gaze based on gaze data that user's gaze data that is input from an outer side and is correlated with the same time axis as in the voice data to analyze the degree of attention (gaze point) of the gaze.

Next, the analysis unit 12a allocates the corresponding gaze period to the gaze data based on the gaze period of the degree of attention analyzed in Step S204 and the important period of the voice data which is set by the setting unit 11 and records the corresponding gaze period in the recording unit 14 (Step S205). Specifically, the analysis unit 12a allocates a value (rank) obtained by multiplying the degree of attention of the voice data before and after the important period by a coefficient (for example, a numerical character of 1 to 9) corresponding to the keyword as the corresponding gaze period, and records the corresponding gaze period in the recording unit 14. According to this, it is possible to analyze the important period in a user's gaze period and it is possible to record the important period in the recording unit 14. After Step S205, the information processing apparatus 10a transitions to Step S206 to be described later. Step S206 and Step S207 respectively correspond to Step S105 and Step S106 in FIG. 2.

FIG. 8B is a view schematically describing a setting method in which the analysis unit 12a sets the degree of importance to the gaze data. In (a) of FIG. 8B, (b) of FIG. 8B, and (c) of FIG. 8B, the horizontal axis represents time, the vertical axis in (a) of FIG. 8B represents the degree of importance of voice, the vertical axis in (b) of FIG. 8B represents a gaze movement speed, and the vertical axis in (c) of FIG. 8B represents the degree of importance of the gaze. In addition, a curved line L2 in FIG. 8B represents a variation of the degree of importance of voice with the passage of time, a curved line L3 in (b) of FIG. 8B represents a variation of the gaze movement speed of the gaze with the passage of time, and a curved line L4 in (c) of FIG. 8B represents a variation of the degree of attention with the passage of time.

Typically, analysis can be made as follows. The greater the movement speed of the gaze is, the lower the degree of attention of a user is. That is, as indicated by the curved line L3 and L4 in FIG. 8B, the analysis unit 12 performs analysis in such a manner that the greater the movement speed of the gaze of a user is, the lower the degree of attention of the gaze of the user is, and the smaller the movement speed of the gaze is (refer to a period D2 in which the movement speed of the gaze is small), the higher the degree of attention of the gaze of the user. As described above, with respect to the gaze data that is user's gaze data input from an outer side and is correlated with the same time axis as in the voice data, the analysis unit 12 allocates the gaze period D2, which is a period before and after an important period D1 in which the degree of importance of voice of the voice data which is set by the setting unit 11 is high and in which the degree of attention of the gaze of the user is high, as the corresponding gaze period (refer to the curved line L4 in (c) of FIG. 8B). Note that, in FIG. 8B, the analysis unit 12 analyzes the degree of attention of a gaze of the user by detecting the movement speed of the gaze of the user, but there is no limitation thereto. The analysis unit 12 may analyze the degree of attention of the gaze by detecting any one of the movement distance of the gaze of the user in a constant time, and a residence time of the gaze of the user in a constant area.

According to the above-described second embodiment, after the analysis unit 12a analyzes the degree of attention of the gaze (gaze point) based on the gaze data that is user's gaze data input from an outer side and is correlated with the same time axis as in the voice data, based on a gaze period for which the degree of attention is analyzed and the important period of the voice data which is set by the setting unit 11, the analysis unit 12a extracts a gaze period for which the degree of attention is analyzed, allocates the corresponding period of the gaze data before and after the important period of the voice data based on the attentioned period and the important period of the voice data which is set by the setting unit 11, and records the corresponding gaze period in the recording unit 14. Accordingly, it is possible to understand the important period in a user's gaze period with respect to the gaze data.

### Third Embodiment

Next, a third embodiment will be described. In the first embodiment in which described a information processing system, the gaze data, the voice data, and the keyword are respectively input from an outer side. However, in the third embodiment, the system incorporates a gaze data and a voice data input unit, and important word information with which the keyword and a coefficient are correlated is recorded in advance. In the following description, processing that is executed by an information processing apparatus according to the third embodiment will be described after describing a configuration of the information processing apparatus according to the third embodiment. Note that, the same reference numeral will be given to the same configuration as in the information processing system 1 according to the first embodiment, and detailed description thereof will be appropriately omitted.

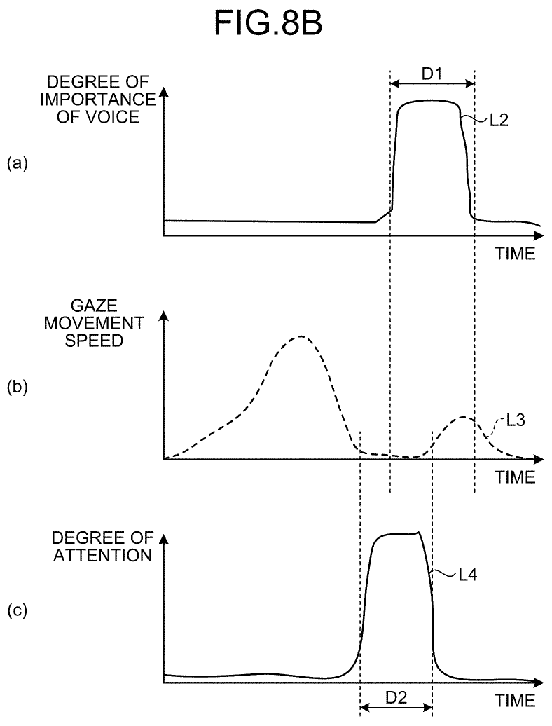

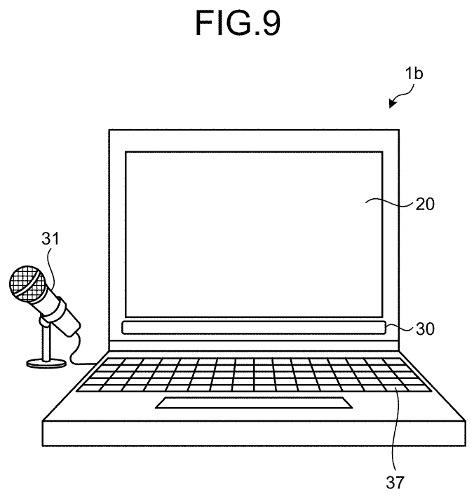

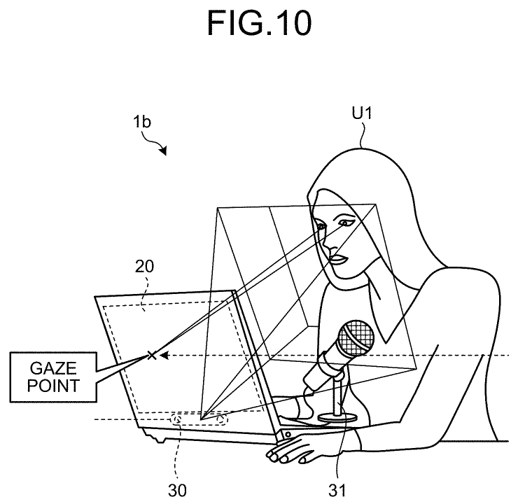

FIG. 9 is a schematic view illustrating a configuration of the information processing apparatus according to the third embodiment. FIG. 10 is a schematic view illustrating the configuration of the information processing apparatus according to the third embodiment. FIG. 11 is a block diagram illustrating a functional configuration of the information processing apparatus according to the third embodiment.

An information processing apparatus 1b illustrated in FIG. 9 to FIG. 11 includes an analysis unit 12, a display unit 20, a gaze detection unit 30, a voice input unit 31, a control unit 32, a time measurement unit 33, a recording unit 34, a converter 35, an extraction unit 36, an operating unit 37, a setting unit 38, a generation unit 39, a program storage unit 344, and an important word storage unit 345.

The gaze detection unit 30 is constituted by using an LED light source that emits near infrared rays, and an optical sensor (for example, CMOS, CCD, or the like) that captures images of a pupil point on the cornea and a reflection point. The gaze detection unit 30 is provided at a lateral surface of a housing of the information processing apparatus 1b at which a user U1 can visually recognize the display unit 20 (refer to FIG. 9 and FIG. 10). The gaze detection unit 30 detects the gaze of the user U1 with respect to an image that is displayed by the display unit 20 under the control of the control unit 32, and outputs the gaze data to the control unit 32. Specifically, the gaze detection unit 30 irradiates near infrared rays emitted from the LED light source or the like, to the cornea of the user U1, under control of the control unit 32. the image of the cornea of user U1, including the pupil and the reflection point on the cornea, is captured with an optical sensor, and send the signal to the control unit

The voice input unit 31 is constituted by using a microphone to which voice is input, a voice codec that converts the voice which the microphone receives input thereof into digital voice data, amplifies the voice data, and outputs the voice data to the control unit 32. The voice input unit 31 receives the input of the voice of the user U1, generates the voice data, and outputs the voice data to the voice input controller 322 under the control of the control unit 32. The control unit 32 is constituted by using a CPU, an FPGA, a GPU, or the like, and controls the gaze detection unit 30, the voice input unit 31, and the display unit 20. The control unit 32 includes a gaze detection controller 321, a voice input controller 322, and a display controller 323.

The gaze detection controller 321 controls the gaze detection unit 30, and receives the signal from the gaze detection unit. Specifically, the gaze detection controller 321 causes the gaze detection unit 30 to irradiate the user U1 with near infrared rays for every predetermined timing, and causes the gaze detection unit 30 to image the pupil of the user U1 to generate the gaze data. The gaze detection controller 321 continuously calculate a gaze of the user U1 from a pattern of the pupil and the reflection point of cornea, based on an analysis result obtained through image processing or the like, to generate gaze data for a predetermined time, and outputs the gaze data to a gaze data recording unit 341. Note that, gaze of the user U1 may detect with using known pattern matching technique with obtained image, or may generate the gaze data by detecting the gaze of the user U1 by using another kind of sensor or another known technology.

The voice input controller 322 controls the voice input unit 31 and receive the voice signal from input unit, may also have various kinds of signal processing with, for example, gain increasing processing, noise reduction processing, and the like respect to the voice data that is input from the voice input unit 31, and outputs the resultant voice data to the recording unit 34.

The display controller 323 controls a display aspect of the display unit 20. The display controller 323 causes the display unit 20 to display an image corresponding to image data that is recorded in the recording unit 34 or a gaze mapping image corresponding to gaze mapping data that is generated by the generation unit 39.

The time measurement unit 33 is constituted by using a timer, a clock generator, or the like, and applies time information with respect to the gaze data generated by the gaze detection unit 30, the voice data generated by the voice input unit 31, and the like.

The recording unit 34 is constituted by using a volatile memory, a nonvolatile memory, a recording medium, or the like, and records various pieces of information related to the information processing apparatus 1b. The recording unit 34 includes a gaze data recording unit 341, a voice data recording unit 342, and an image data recording unit 343.

The gaze data recording unit 341 records the gaze data that is input from the gaze detection controller 321, and outputs the gaze data to the analysis unit 12.

The voice data recording unit 342 records the voice data that is input from the voice input controller 322, and outputs the voice data to the converter 35.

The image data recording unit 343 records a plurality of pieces of image data. The plurality of pieces of image data include data that is input from an outer side of the information processing apparatus 1b, or data that is imaged by an imaging device on an outer side in accordance with a recording medium.

The converter 35 performs known text conversion processing with respect to the voice data to convert the voice data into textual information (text data), and outputs the textual information to the extraction unit 36. Note that, the conversion of voice into characters may not performed at this point of time, and in this case, the degree of importance may be set in the voice information state as is, and then conversion into the textual information may be performed.

The extraction unit 36 extracts a keyword (a word or characters) corresponding to an instruction signal that is input from the operating unit 37 to be described later, or a plurality of keywords which are recorded by the important word storage unit 345 to be described later from the textual information that is converted by the converter 35, and outputs the extraction result to the setting unit 38.

The operating unit 37 is constituted by using a mouse, a keyboard, a touch panel, various switches, or the like, receives an operation input of the user U1, and outputs the operation content, of which input is received, to the control unit 32.

The setting unit 38 sets a period in which the keyword extracted by the extraction unit 36 is uttered in the voice data as an important period, and outputs the setting result to the analysis unit 12.

The generation unit 39 generates gaze mapping data in which the corresponding gaze period analyzed by the analysis unit 12 and the textual information converted by the converter 35 are correlated with an image corresponding to the image data that is displayed by the display unit 20, and outputs the gaze mapping data to the image data recording unit 343 or the display controller 323.

The program storage unit 344 records various programs which are executed by the information processing apparatus 1b, data (for example, dictionary information or text conversion dictionary information) that is used during execution of the various programs, and processing data during execution of the various programs.

The important word storage unit 345 records important word information with which a plurality of keywords and an index are correlated. For example, in the important word storage unit 345, in a case where a keyword is “cancer”, “10” is correlated as the index, and in a case where the keyword is “bleeding”, “8” is correlated as the index, and in a case where the keyword is “without abnormality”, “0” is correlated as the index.

Processing of Information Processing Apparatus

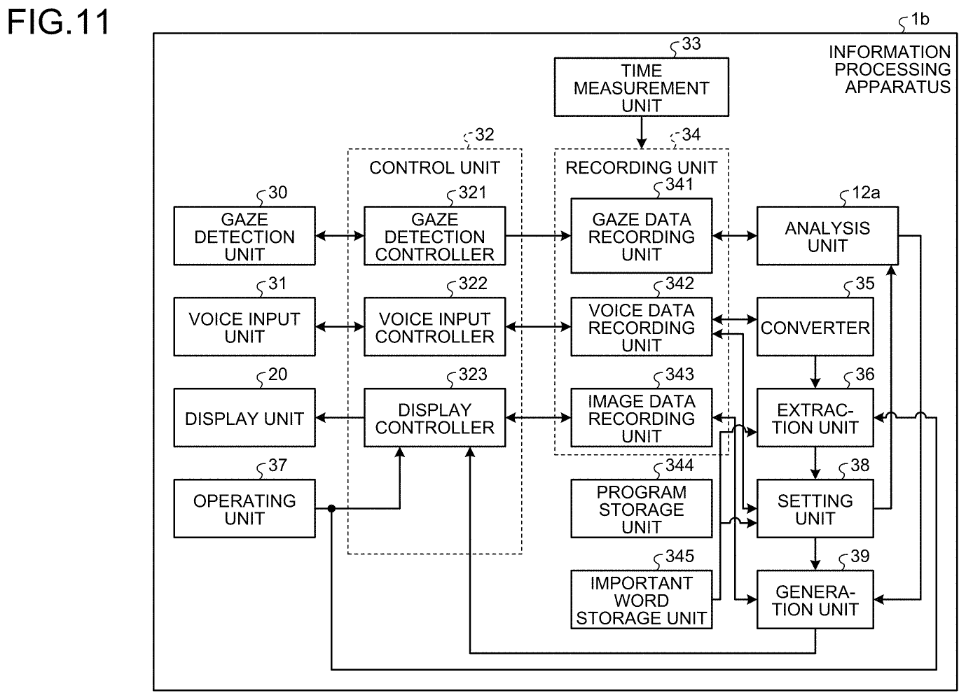

Next, processing that is executed by the information processing apparatus 1b will be described. FIG. 12 is a flowchart illustrating an outline of processing that is executed by the information processing apparatus 1b.

As illustrated in FIG. 12, first, the display controller 323 causes the display unit 20 to display an image corresponding to the image data that is recorded by the image data recording unit 343 (Step S301). In this case, the display controller 323 causes the display unit 20 to display an image corresponding to image data that is selected in accordance with an operation of the operating unit 37.

Next, the control unit 32 records the gaze data generated by the gaze detection unit 30 and the voice data generated by the voice input unit 31 in the gaze data recording unit 341 and the voice data recording unit 342, respectively, in correlation with time measured by the time measurement unit 33 (Step S302).

Then, the converter 35 converts the voice data that is recorded in the voice data recording unit 342 into textual information (Step S303). Note that, the step may be performed after S308 to be described later.

Next, in a case where it is determined that an instruction signal indicating termination of observation of the image that is displayed by the display unit 20 is input from the operating unit 37 (Step S304: Yes), the information processing apparatus 1b transitions to Step S305 to be described later. In contrast, in a case where it is determined that the instruction signal indicating termination of observation of the image that is displayed by the display unit 20 is not input from the operating unit 37 (Step S304: No), the information processing apparatus 1b returns to Step S302.

Step S305 to Step S308 respectively corresponds to Step S202 to Step S205 in FIG. 8A. After Step S308, the information processing apparatus 1b transitions to Step S309 to be described later.

Next, the generation unit 39 generates gaze mapping data in which the corresponding gaze period analyzed by the analysis unit 12 and the textual information converted by the converter 35 are correlated with an image corresponding to the image data that is displayed by the display unit 20 (Step S309).

Next, the display controller 323 causes the display unit 20 to display a gaze mapping image corresponding to the gaze mapping data that is generated by the generation unit 39 (Step S310).

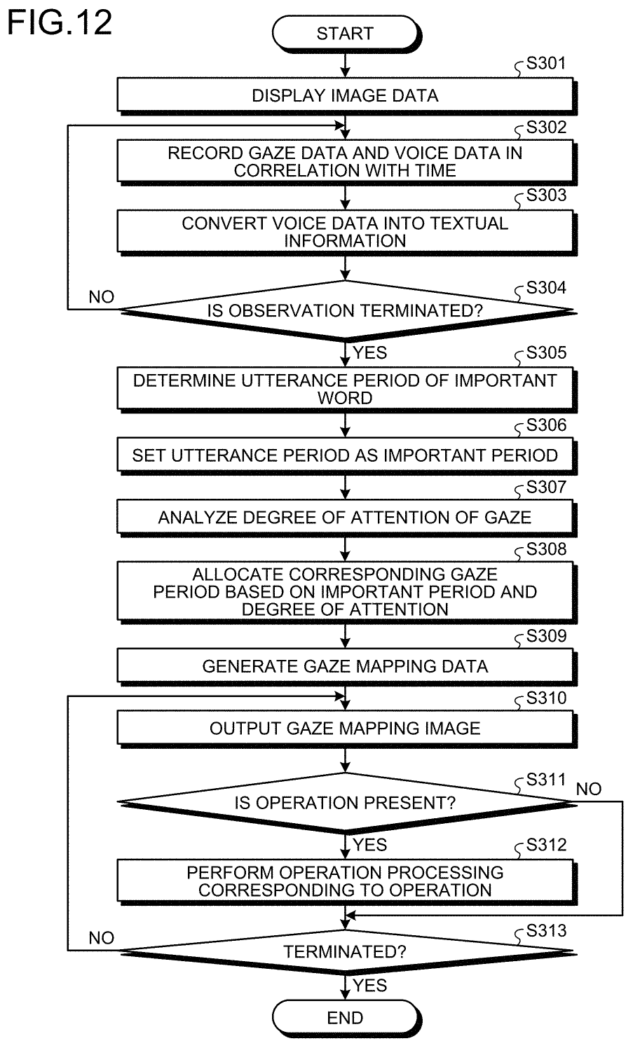

FIG. 13 is a view illustrating an example of the gaze mapping image that is displayed by the display unit 20. As illustrated in FIG. 13, the display controller 323 causes the display unit 20 to display a gaze mapping image P3 corresponding to the gaze mapping data that is generated by the generation unit 39. Records M11 to M15 corresponding to gaze areas of a gaze based on the rank of the corresponding gaze period, and a trajectory K1 of the gaze are superimposed on the gaze mapping image P3, and textual information of the voice data that is uttered at timing of the corresponding gaze period is correlated with the gaze mapping image P3. In addition, in the records M11 to M15, the number thereof represents the order of the gaze of the user U1, and a size (area) represents the magnitude of the rank of the corresponding gaze period. In addition, in a case where the user U1 operates the operating unit 37 to move a cursor Al to a desired position, for example, to the record M14, a message Q1 that is correlated with the record M14, for example, “here is cancer” is displayed. Note that, in FIG. 13, the display controller 323 causes the display unit 20 to display the textual information, but may output voice data after converting the textual information into voice as an example. According to this, the user U1 can intuitively understand content that is uttered with voice and a gazing area. In addition, it is possible to intuitively understand a trajectory of the gaze during observation of the user U1.

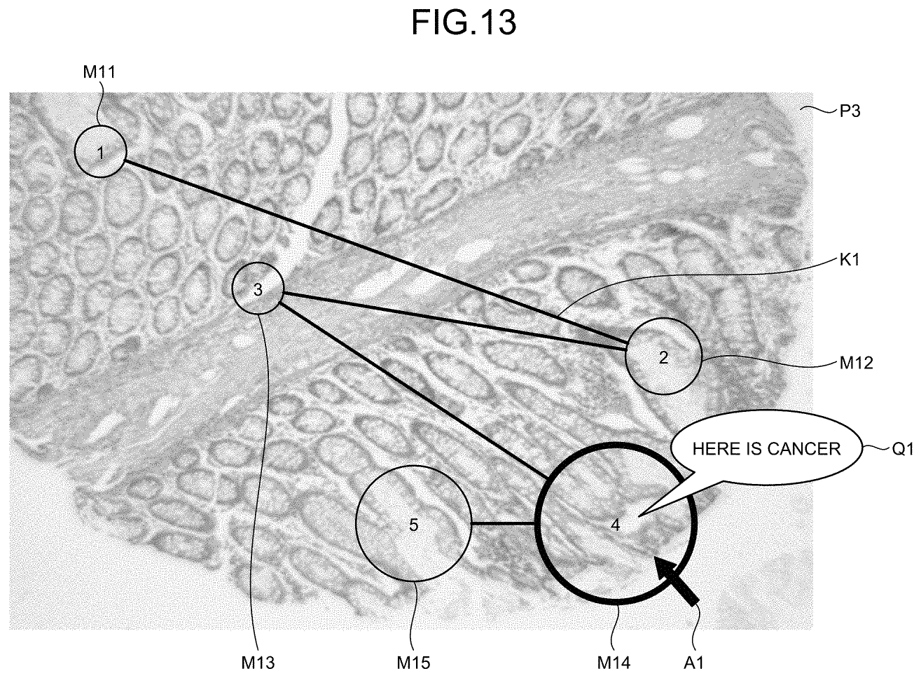

FIG. 14 is a view illustrating another example of the gaze mapping image that is displayed by the display unit 20. As illustrated in FIG. 14, the display controller 323 causes the display unit 20 to display a gaze mapping image P4 corresponding to the gaze mapping data that is generated by the generation unit 39. In addition, the display controller 323 causes the display unit 20 to display icons B1 to B5 in which textual information and time at which the textual information is uttered are correlated. In addition, in a case where the user U1 operates the operating unit 37 and selects any one of the records M11 to M15, for example, the record M14 is selected, the display controller 323 highlights the record M14 on the display unit 20, and highlights textual information corresponding to time of the record M14, for example, the icon B4 on the display unit 20 (for example, a frame is highlighted or is displayed with a bold line). According to this, the user U1 can intuitively understand important voice content and a gazing area, and can intuitively understand content at the time of utterance.

Returning to FIG. 12, description of processing subsequent to Step S311 will continue.

In Step S311, in a case where it is determined that any one of the records corresponding to a plurality of gaze areas is operated by the operating unit 37 (Step S311: Yes), the control unit 32 executes operation processing corresponding to the operation (Step S312). Specifically, the display controller 323 causes the display unit 20 to highlight a record corresponding to the gaze area that is selected by the operating unit 37 (for example, refer to FIG. 13). In addition, the voice input controller 322 causes the voice input unit 31 to reproduce voice data that is correlated with an area of which the degree of attention is high. After Step S312, the information processing apparatus 1b transitions to Step S313 to be described later.

In Step S311, in a case where it is determined that any one of the records corresponding to the plurality of dgaze areas is not operated by the operating unit 37 (Step S311: No), the information processing apparatus 1b transitions to Step S313 to be described later.

In Step S313, in a case where it is determined that the instruction signal indicating termination of observation is input from the operating unit 37 (Step S313: Yes), the information processing apparatus 1b terminates the processing. In contrast, in a case where it is determined that the instruction signal indicating termination of observation is not input from the operating unit 37 (Step S313: No), the information processing apparatus 1b returns to Step S310 as described above.

According to the above-described third embodiment, since the generation unit 39 generates gaze mapping data in which the corresponding gaze period analyzed by the analysis unit 12 and the textual information converted by the converter 35 are correlated with an image corresponding to the image data that is displayed by the display unit 20, the user U1 can intuitively understand content of the corresponding gaze period and a gazing area, and it is possible to intuitively understand content at the time of utterance.

In addition, according to the third embodiment, since the display controller 323 causes the display unit 20 to display the gaze mapping image corresponding to the gaze mapping data generated by the generation unit 39, the present disclosure can be used in confirmation of prevention of observation overlooking of a user with respect to an image, confirmation of a technology skill such as interpretation of a user, teaching of interpretation, observation, or the like with respect to another user, a conference, and the like.

### Fourth Embodiment

Next, a fourth embodiment will be described. In the third embodiment, only the information processing apparatus 1b is provided, but in the fourth embodiment, an information processing apparatus is combined to a part of a microscopic system. In the following description, processing that is executed by the microscopic system according to the fourth embodiment will be described after describing a configuration of the microscopic system according to the fourth embodiment. Note that, the same reference numeral will be given to the same configuration as in the information processing apparatus 1b according to the third embodiment, and detailed description thereof will be appropriately omitted.

Configuration of Microscopic System

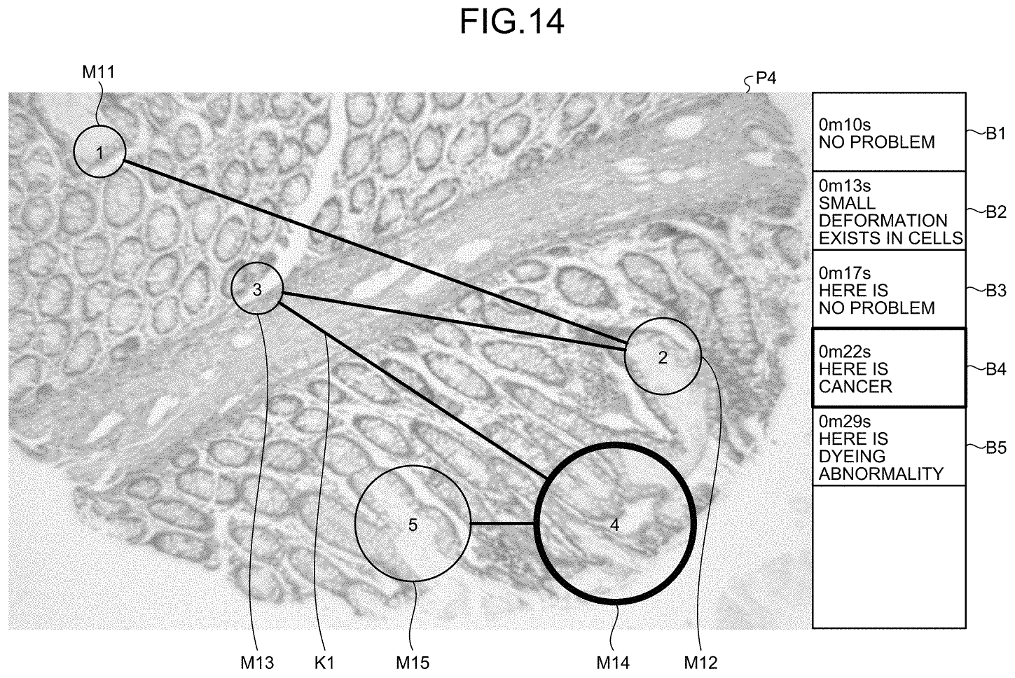

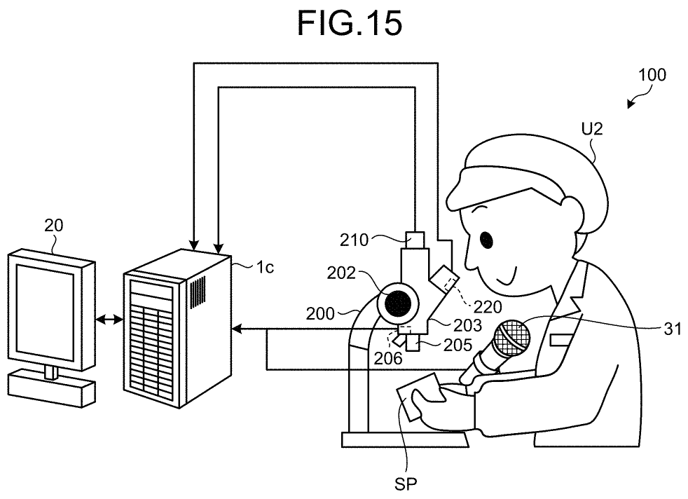

FIG. 15 is a schematic view illustrating a configuration of the microscopic system according to the fourth embodiment. FIG. 16 is a block diagram illustrating a functional configuration of the microscopic system according to the fourth embodiment.

As illustrated in FIG. 15 and FIG. 16, a microscopic system 100 includes an information processing apparatus 1c, a display unit 20, a voice input unit 31, an operating unit 37, a microscope 200, an imaging unit 210, and a gaze detection unit 220.

Configuration of Microscope

First, a configuration of the microscope 200 will be described.

The microscope 200 includes a main body portion 201, a rotary portion 202, an elevating portion 203, a revolver 204, an objective lens 205, a magnification detection portion 206, a lens barrel portion 207, a connection portion 208, and an eyepiece portion 209.

A specimen SP is placed on the main body portion 201. The main body portion 201 has an approximately U-shape and is connected to the elevating portion 203 by using the rotary portion 202.

The rotary portion 202 rotates in accordance with an operation of a user U2 and moves the elevating portion 203 in a vertical direction.

The elevating portion 203 is provided to move in a vertical direction with respect to the main body portion 201. A revolver 204 is connected a surface on one end side of the elevating portion 203, and the lens barrel portion 207 is connected to a surface on the other side thereof.

A plurality of the objective lenses 205 of which magnifications are different from each other are connected to the revolver 204, and the revolver 204 is connected to the elevating portion 203 in a rotatable manner with respect to an optical axis L1. The revolver 204 disposes a desired objective lens 205 on the optical axis L1 in accordance with an operation of the user U2. Note that, information indicating the magnification, for example, an IC chip or a label is attached to the plurality of objective lenses 205. Note that, in addition to the IC chip or the label, a shape indicating the magnification may be formed in the objective lenses 205.

The magnification detection portion 206 detects the magnifications of the objective lens 205 that is placed on the optical axis L1, and outputs the detection result to the information processing apparatus 1c. For example, the magnification detection portion 206 is constituted by using a unit that detects a position of the revolver 204 for objective switching.

The lens barrel portion 207 allows a part of a subject image of the specimen SP which is formed by the objective lens 205 to be transmitted therethrough the connection portion 208, and reflects the part to the eyepiece portion 209. The lens barrel portion 207 includes a prism, a semi-transparent mirror, a collimate lens, and the like on an inner side.

In the connection portion 208, one end is connected to the lens barrel portion 207, and the other end is connected to the imaging unit 210. The connection portion 208 guides the subject image of the specimen SP which is transmitted through the lens barrel portion 207 to the imaging unit 210. The connection portion 208 is constituted by using a plurality of the collimate lenses and the imaging lenses, and the like.

The eyepiece portion 209 guides the subject image reflected by the lens barrel portion 207 and forms an image. The eyepiece portion 209 is constituted by using a plurality of the collimate lenses and the imaging lenses, and the like.

Configuration of Imaging Unit

Next, a configuration of the imaging unit 210 will be described.

The imaging unit 210 receives the subject image of the specimen SP which is formed by the connection portion 208 to generate image data, and outputs the image data to the information processing apparatus 1c. The imaging unit 210 is constituted by using an image sensor such as a CMOS and a CCD, an image processing engine that performs various kinds of image processing with respect to the image data, and the like.

Configuration of Gaze Detection Unit

Next, a configuration of the gaze detection unit 220 will be described.

The gaze detection unit 220 is provided on an inner side or an outer side of the eyepiece portion 209, generates gaze data by detecting a gaze of the user U2, and outputs the gaze data to the information processing apparatus 1c. The gaze detection unit 220 is constituted by using an LED light source that is provided on an inner side of the eyepiece portion 209 and emits near infrared rays, and an optical sensor (for example, a CMOS or a CCD) that is provided on an inner side of the eyepiece portion 209 and captures images of a pupil point on the cornea and a reflection point. The gaze detection unit 220 irradiates the cornea of the user U2 with near infrared rays emitted from the LED light source or the like under control of the information processing apparatus 1c, and the optical sensor captures images of a pupil point on the cornea and a reflection point of the user U2 to generate the gaze data. In addition, a gaze detection unit 220 generates gaze data by detecting the gaze of the user from a pattern of the pupil point of the user U2 and the reflection point based on an analysis result obtained through analysis performed by imaging processing or the like with respect to the data generated by the optical sensor under control of the information processing apparatus 1c, and outputs the gaze data to the information processing apparatus 1c.

Configuration of Information Processing Apparatus

Next, a configuration of the information processing apparatus 1c will be described.

The information processing apparatus 1c includes a control unit 32c, a recording unit 34c, and an analysis unit 40 in substitution for the control unit 32, the recording unit 34, and the analysis unit 12 of the information processing apparatus 1b according to the third embodiment.

The control unit 32c is constituted by using a CPU, an FPGA, a GPU, or the like, and controls the display unit 20, the voice input unit 31, the imaging unit 210, and the gaze detection unit 220. The control unit 32c further includes an imaging controller 324 and a magnification calculation unit 325 in addition to the gaze detection controller 321, the voice input controller 322, and the display controller 323 of the control unit 32 of the third embodiment.

The imaging controller 324 controls an operation of the imaging unit 210. The imaging controller 324 causes the imaging unit 210 to sequentially perform imaging in accordance with a predetermined frame rate to generate image data. The imaging controller 324 performs predetermined image processing (for example, development processing or the like) with respect to the image data that is input from the imaging unit 210, and outputs the resultant image data to the recording unit 34c.

The magnification calculation unit 325 calculates a current observation magnification of the microscope 200 based on a detection result that is input from the magnification detection portion 206, and outputs the calculation result to the analysis unit 40. For example, the magnification calculation unit 325 calculates the current observation magnification of the microscope 200 based on a magnification of the objective lens 205 and a magnification of the eyepiece portion 209 which are input from the magnification detection portion 206.

The recording unit 34c is constituted by using a volatile memory, a nonvolatile memory, a recording medium, or the like. The recording unit 34c includes an image data recording unit 346 in substitution for the image data recording unit 343 according to the third embodiment. The image data recording unit 346 records the image data that is input from the imaging controller 324, and outputs the image data to the generation unit 39.

The analysis unit 40 analyzes the degree of attention of a gaze (gaze point) by detecting any one of a movement speed of the gaze, a movement distance of the gaze in a constant time, and a residence time of the gaze in a constant area based on the gaze data that is correlated with the same time axis as in the voice data. In addition, the analysis unit 40 allocates the corresponding gaze period and the textual information converted by the converter 35 to the gaze data based on the gaze period of the degree of attention that is analyzed, the important period of the voice data which is set by the setting unit 38, and the calculation result calculated by the magnification calculation unit 325, and records the corresponding gaze period and the textual information in the recording unit 34c. Specifically, the analysis unit 40 allocates a value, which is obtained by multiplying the gaze period of the degree of attention that is analyzed by a coefficient based on the calculation result calculated by the magnification calculation unit 325 and a coefficient corresponding to a keyword of the important period set by the setting unit 38, to the gaze period (time) of the degree of attention of the gaze data which corresponds to a period before and after the important period of the voice data and the corresponding gaze period, and records the corresponding gaze period on the recording unit 34c. That is, the analysis unit 40 performs processing so that the greater a display magnification is, the higher the rank of the corresponding gaze period becomes. A setting unit 38c is constituted by using a CPU, an FPGA, a GPU, or the like.

Processing of Microscopic System

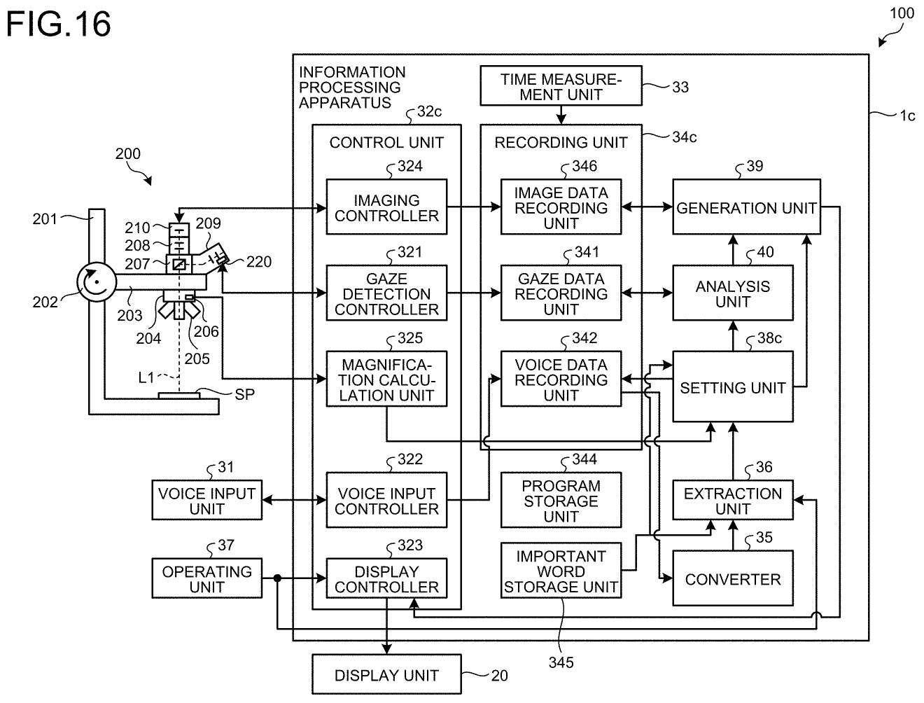

Next, processing that is executed by the microscopic system 100 will be described. FIG. 17 is a flowchart illustrating an outline of the processing that is executed by the microscopic system 100.

As illustrated in FIG. 17, first, the control unit 32c records the gaze data generated by the gaze detection unit 30, the voice data generated by the voice input unit 31, and the observation magnification calculated by the magnification calculation unit 325 in the gaze data recording unit 341 and the voice data recording unit 342 in correlation with time measured by the time measurement unit 33 (Step S401). After Step S401, the microscopic system 100 transitions to Step S402 to be described later.

Step S402 to Step S406 respectively corresponds to Step S302 to Step S307 in FIG. 12. After Step S406, the microscopic system 100 transitions to Step S407.

In Step S407, the analysis unit 40 allocates the corresponding gaze period and the textual information converted by the converter 35 to the gaze data based on the degree of attention that is analyzed, the important period of the voice data which is set by the setting unit 11, and the calculation result calculated by the magnification calculation unit 325, and records the corresponding gaze period and the textual information in the recording unit 34c. Specifically, the analysis unit 40 allocates a value, which is obtained by multiplying the degree of attention that is analyzed by a coefficient based on the calculation result calculated by the magnification calculation unit 325 and a coefficient corresponding to a keyword of the important period, to the gaze period (time) of the degree of attention of the gaze data corresponding to a period before and after the important period of the voice data as the corresponding gaze period, and the records the corresponding gaze period in the recording unit 34c. After Step S407, the microscopic system 100 transitions to Step S408.

Step S408 to Step S412 respectively corresponds to Step S309 to Step S313 in FIG. 12.

According to the above-described fourth embodiment, since the setting unit 38c allocates the degree of importance and the textual information converted by the converter 35 to the voice data that is correlated with the same time axis as in the gaze data based on the degree of attention that is analyzed by the analysis unit 40 and the calculation result calculated by the magnification calculation unit 325, and records the degree of importance and the textual information in the recording unit 34c, the degree of importance based on the observation magnification and the degree of attention is allocated to the voice data. Accordingly, it is possible to understand the important period of the voice data in consideration of the observation content and the degree of attention.

Note that, in the fourth embodiment, the observation magnification calculated by the magnification calculation unit 325 is recorded in the recording unit 14. However, an operation history of the user U2 may be recorded, and the corresponding gaze period of the gaze data may be allocated by adding the operation history thereto.

### Fifth Embodiment

Next, a fifth embodiment will be described. In the fifth embodiment, an information processing apparatus is combined to a part of an endoscopic system. In the following description, processing that is executed by the endoscopic system according to the fourth embodiment will be described after describing a configuration of the endoscopic system according to the fifth embodiment. Note that, the same reference numeral will be given to the same configuration as in the information processing apparatus 1b according to the third embodiment, and detailed description thereof will be appropriately omitted.

Configuration of Endoscopic System

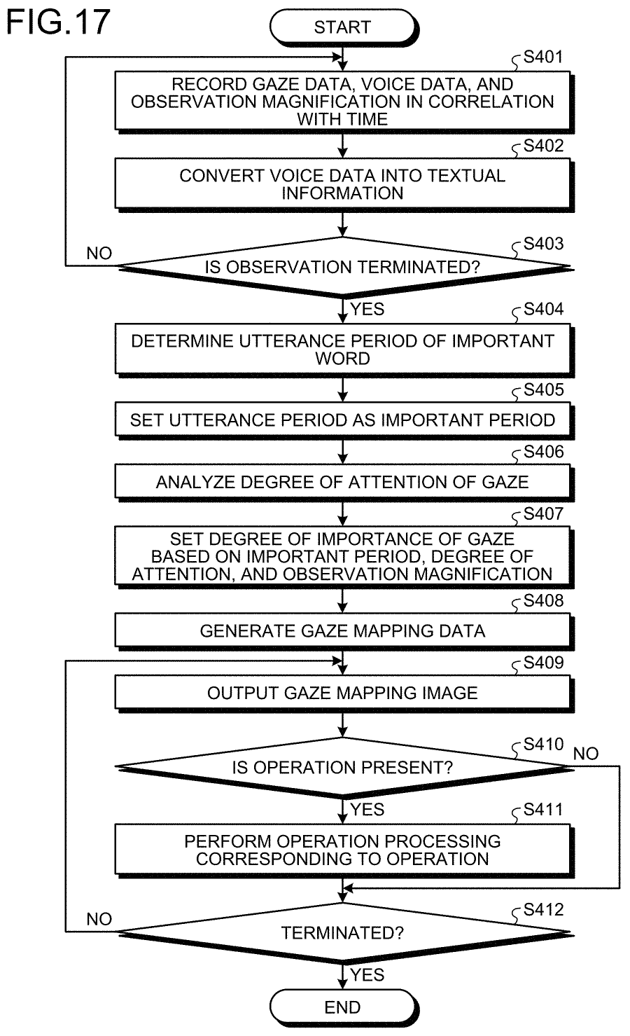

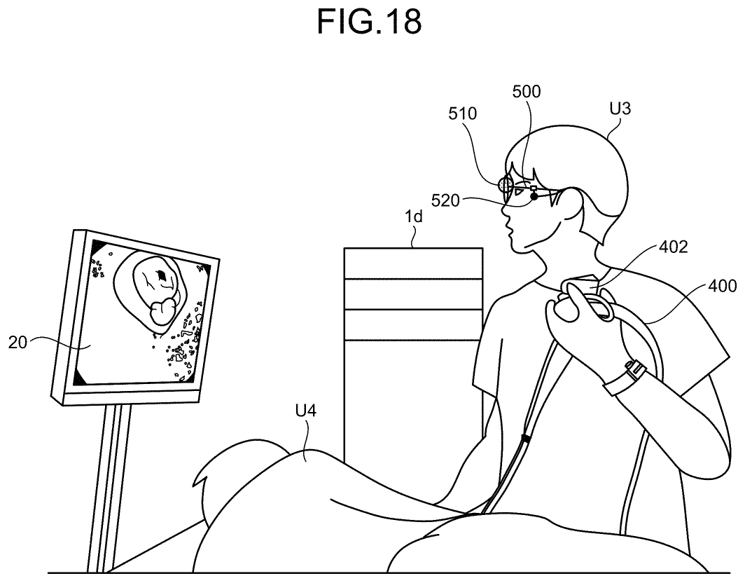

FIG. 18 is a schematic view illustrating the configuration of the endoscopic system according to the fifth embodiment. FIG. 19 is a block diagram illustrating a functional configuration of the endoscopic system according to the fifth embodiment.

An endoscopic system 300 illustrated in FIG. 18 and FIG. 19 includes the display unit 20, an endoscope 400, a wearable device 500, an input unit 600, and an information processing apparatus 1d.

Configuration of Endoscope

First, a configuration of the endoscope 400 will be described.

The endoscope 400 is inserted into a subject U4 by a user U3 such as a doctor and operator, captures images of the inside of the subject U4 to generate image data, and outputs the image data to the information processing apparatus 1d. The endoscope 400 includes an imaging unit 401 and an operating unit 402.

The imaging unit 401 is provided at a distal end of an insertion portion of the endoscope 400. The imaging unit 401 captures images of the inside of the subject U4 under control of the information processing apparatus 1d to generate image data, and outputs the image data to the information processing apparatus 1d. The imaging unit 401 is constituted by using an optical system capable of changing an observation magnification, an image sensor such as a CMOS and a CCD that receives a subject image that is formed by the optical system to generate image data, and the like.

The operating unit 402 receives inputs of various operations of the user U3, and outputs operation signals corresponding to the various operations which are received to the information processing apparatus 1d.

Configuration of Wearable Device

Next, a configuration of the wearable device 500 will be described.

The wearable device 500 is worn on the user U3 to detect a gaze of the user U3 and to receive an input of voice of the user U3. The wearable device 500 includes a gaze detection unit 510 and a voice input unit 520.

The gaze detection unit 510 is provided in the wearable device 500, detects the degree of attention of the gaze of the user U3 to generate gaze data, and outputs the gaze data to the information processing apparatus 1d. The gaze detection unit 510 has the same configuration as the gaze detection unit 220 according to the fourth embodiment, and thus detailed description thereof will be appropriately omitted.

The voice input unit 520 is provided in the wearable device 500, receives input of voice of the user U3 to generate voice data, and outputs the voice data to the information processing apparatus 1d. The voice input unit 520 is constituted by using a microphone or the like.

Configuration of Input Unit

A configuration of the input unit 600 will be described.

The input unit 600 is constituted by using a mouse, a keyboard, a touch panel, and various switches. The input unit 600 receives inputs of various operations of the user U3, and outputs operation signals corresponding to various operations which are received to the information processing apparatus 1d.

Configuration of Information Processing Apparatus

Next, a configuration of the information processing apparatus 1d will be described.

The information processing apparatus 1d includes a control unit 32d, a recording unit 34d, a setting unit 38d, and an analysis unit 40d in substitution for the control unit 32c, the recording unit 34c, the setting unit 38c, and the analysis unit 40 of the information processing apparatus 1c according to the fourth embodiment. In addition, the information processing apparatus 1d further includes an image processing unit 41.

The control unit 32d is constituted by using a CPU, an FPGA, a GPU, or the like, and controls the endoscope 400, the wearable device 500, and the display unit 20. The control unit 32d includes an operation history detection unit 326 in addition to the gaze detection controller 321, the voice input controller 322, the display controller 323, and the imaging controller 324.

The operation history detection unit 326 detects content of the operation of which input is received by the operating unit 402 of the endoscope 400, and outputs the detection result to the recording unit 34d. Specifically, in a case where an enlargement switch is operated form the operating unit 402 of the endoscope 400, the operation history detection unit 326 detects the operation content and outputs the detection result to the recording unit 34d. Note that, the operation history detection unit 326 may detect operation content of a treatment tool that is inserted into the subject U4 through the endoscope 400, and may output the detection result to the recording unit 34d.

The recording unit 34d is constituted by using a volatile memory, a nonvolatile memory, a recording medium, or the like. The recording unit 34d further includes an operation history recording unit 347 in addition to the configuration of the recording unit 34c according to the fourth embodiment.

The operation history recording unit 347 records a history of an operation with respect to the operating unit 402 of the endoscope 400 which is input from the operation history detection unit 326.

A generation unit 39d generates gaze mapping data in which the corresponding gaze period analyzed by the analysis unit 40d to be described later and the textual information are correlated with an integrated image corresponding to integrated image data that is generated by the image processing unit 41 to be described later, and outputs the gaze mapping data that is generated to the recording unit 34d and the display controller 323.

The analysis unit 40d analyzes the degree of attention of a gaze (gaze point) by detecting any one of a movement speed of the gaze, a movement distance of the gaze in a constant time, and a residence time of the gaze in a constant area based on the gaze data that is correlated with the same time axis as in the voice data and. In addition, the analysis unit 40d allocates the corresponding gaze period and the textual information converted by the converter 35 to the gaze data based on the degree of attention that is analyzed, the important period of the voice data which is set by the setting unit 38, and the operation history that is recorded by the operation history recording unit 347, and records the corresponding gaze period and the textual information in the recording unit 34d. Specifically, the analysis unit 40d allocates a value, which is obtained by multiplying the degree of attention that is analyzed by a coefficient based on the operation history that is recorded by the operation history recording unit 347 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34d. That is, the analysis unit 40 performs processing so that the greater important operation content such as enlargement observation and treatment countermeasure with respect to a lesion is, the higher the rank of the corresponding gaze period is. The analysis unit 40d is constituted by using a CPU, an FPGA, a GPU, or the like.

The image processing unit 41 synthesizes a plurality of pieces of image data which are recorded by the image data recording unit 346 to generate integrated image data of a three-dimensional image, and outputs the integrated image data to the generation unit 39d.

Processing of Endoscopic System

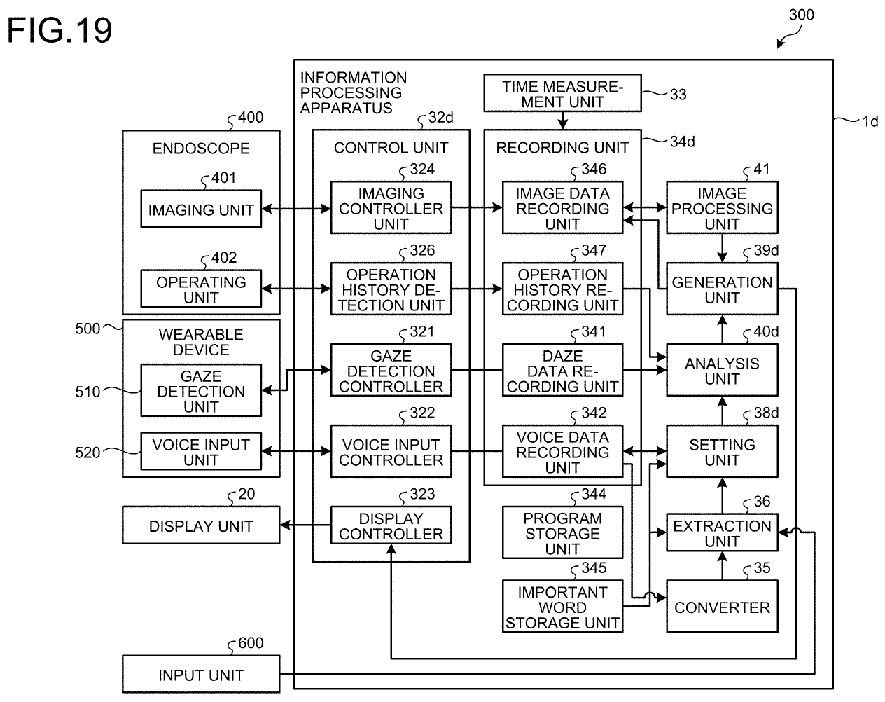

Next, processing that is executed by the endoscopic system 300 will be described. FIG. 20 is a flowchart illustrating an outline of the processing that is executed by the endoscopic system 300.

As illustrated in FIG. 20, first, the control unit 32d records the gaze data generated by the gaze detection unit 30, the voice data generated by the voice input unit 31, and the operation history detected by the operation history detection unit 326 in the gaze data recording unit 341, the voice data recording unit 342, and the operation history recording unit 347 in correlation with time that is measured by the time measurement unit 33 (Step S501). After Step S501, the endoscopic system 300 transitions to Step S502 to be described later.

Step S502 to Step S506 respectively corresponds to Step S303 to Step S307 in FIG. 12. After Step S506, the endoscopic system 300 transitions to Step S507.

In Step S507, the analysis unit 40d allocates the corresponding gaze period and the textual information converted by the converter 35 to the gaze data based on the degree of attention that is analyzed, the important period of the voice data which is set by the setting unit 38, and the operation history that is recorded by the operation history recording unit 347, and records the corresponding gaze period and the textual information in the recording unit 34d. Specifically, the analysis unit 40d allocates a value, which is obtained by multiplying the degree of attention that is analyzed by a coefficient based on the operation history that is recorded by the operation history recording unit 347 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34d.

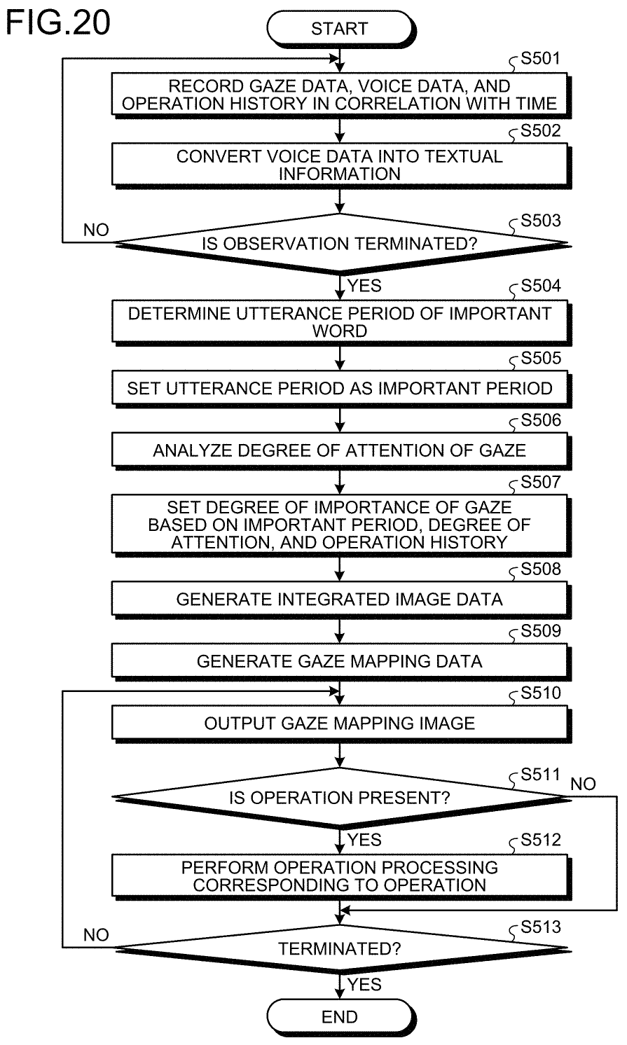

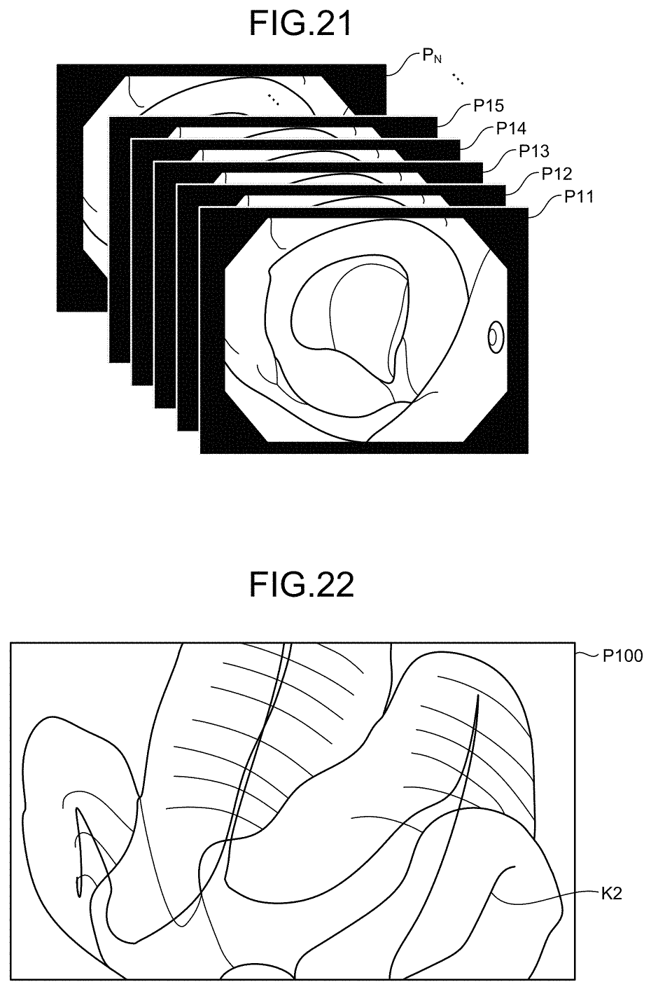

Next, the image processing unit 41 synthesizes a plurality of pieces of image data which are recorded by the image data recording unit 346 to generate integrated image data of a three-dimensional image, and outputs the integrated image data to the generation unit 39d (Step S508). FIG. 21 is a view schematically illustrating an example of a plurality of images which correspond to the plurality of pieces of image data which are recorded by the image data recording unit 346. FIG. 22 is a view illustrating an example of an integrated image corresponding to integrated image data that is generated by the image processing unit 41. As illustrated in FIG. 21 and FIG. 22, the image processing unit 41 synthesizes temporally continuous a plurality of pieces of image data P11 to P_{N} (N is an integer) to generate an integrated image P100 corresponding to the integrated image data.

Then, the generation unit 39d generates gaze mapping data in which the corresponding gaze period analyzed by the analysis unit 40d, gaze, and textual information are correlated with the integrated image P100 corresponding to the integrated image data that is generated by the image processing unit 41, and outputs the gaze mapping data that is generated to the recording unit 34d and the display controller 323 (Step S509). In this case, the generation unit 39d may correlate an operation history with the integrated image P100 corresponding to the integrated image data generated by the image processing unit 41 in addition to the corresponding gaze period analyzed by the analysis unit 40d, the gaze K2, and the textual information. After Step S509, the endoscopic system 300 transitions to Step S510 to be described later.

Step S510 to Step S513 respectively corresponds to Step S310 to Step S313 in FIG. 12.

According to the above-described fifth embodiment, the analysis unit 40d allocates a value, which is obtained by multiplying the degree of attention that is analyzed by a coefficient based on the operation history that is recorded by the operation history recording unit 347 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34d. Accordingly, it is possible to understand the important period of the gaze data in consideration of the operation content and the degree of attention.

In addition, in the fifth embodiment, the endoscopic system has been described, but application is also possible to a capsule-type endoscope, a video microscope that captures images of a subject, a portable telephone provided with an imaging function, and a tablet type terminal provided with the imaging function as an example.

In addition, in the fifth embodiment, the endoscopic system including a soft endoscope has been described, but application is also possible to an endoscopic system including a hard endoscope, and an endoscopic system including an industrial endoscope.

In addition, in the fifth embodiment, the endoscopic system including an endoscope that is inserted into a subject has been described, but application is also possible to endoscopic systems such as a paranasal sinus endoscope, an electric scalpel, and an inspection probe.

### Sixth Embodiment

Next, a sixth embodiment will be described. In the above-described first to fifth embodiments, it is assumed that a user is one person, but in the sixth embodiment, two or more users are assumed. In addition, in the sixth embodiment, an information processing apparatus is combined to an information processing system in which a plurality of users browse an image. In the following description, processing that is executed by the information processing system according to sixth embodiment will be described after describing a configuration of a browsing system according to the sixth embodiment. Note that, the same reference numeral will be given to the same configuration as in the information processing apparatus 1b according to the third embodiment, and detailed description thereof will be appropriately omitted.

Configuration of Information Processing System

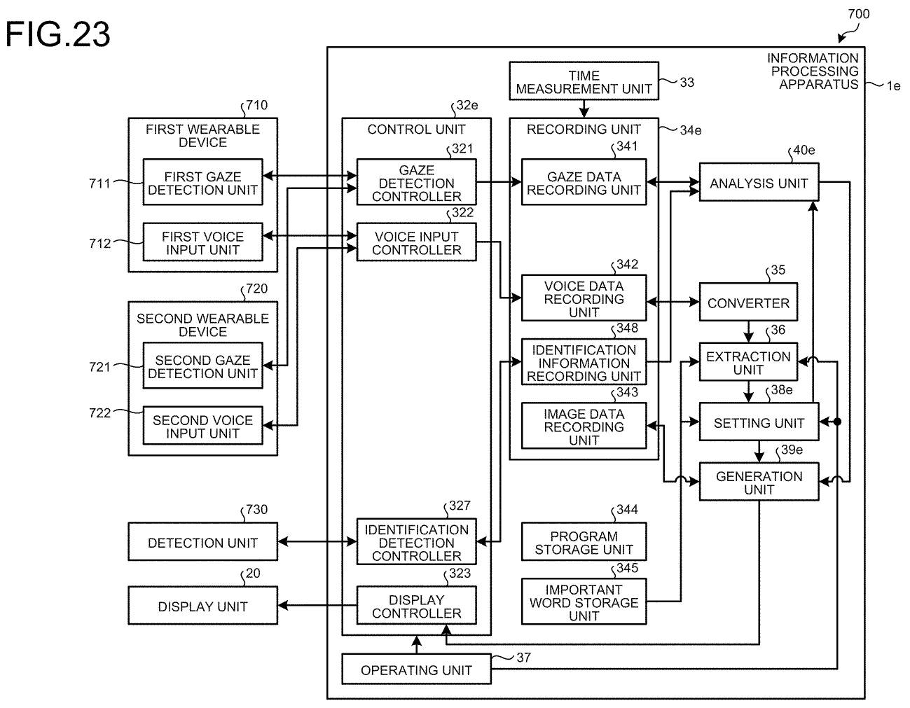

FIG. 23 is a block diagram illustrating a functional configuration of the information processing system according to the sixth embodiment. An information processing system 700 illustrated in FIG. 23 includes the display unit 20, a first wearable device 710, a second wearable device 720, a detection unit 730, and an information processing apparatus 1e.

Configuration of First Wearable Device

First, a configuration of the first wearable device 710 will be described.

The first wearable device 710 is worn on a user, detects a gaze of the user, and receives an input of voice of the user. The first wearable device 710 includes a first gaze detection unit 711 and a first voice input unit 712. The first gaze detection unit 711 and the first voice input unit 712 have a similar configuration as in the gaze detection unit 510 and the voice input unit 520 according to the fifth embodiment, and thus explanation for the detailed configuration thereof will be omitted.

Configuration of Second Wearable Device

Next, a configuration of the second wearable device 720 will be described.

The second wearable device 720 has a similar configuration as in the first wearable device 710, and is worn on a user to detect a gaze of the user and to receive an input of voice of the user. The second wearable device 720 includes a second gaze detection unit 721 and a second voice input unit 722. The second gaze detection unit 721 and the second voice input unit 722 have a similar configuration as in the gaze detection unit 510 and the voice input unit 520 according to the fifth embodiment, and thus explanation for the detailed configuration thereof will be omitted.

Configuration of Detection Unit

Next, a configuration of the detection unit 730 will be described.

The detection unit 730 detects identification information for identifying each of a plurality of users, and outputs the detection result to the information processing apparatus le. The detection unit 730 detects identification information of a user from an IC card that records identification information (for example, ID, name, or the like) for identifying each of the plurality of users, and outputs the detection result to the information processing apparatus le. For example, the detection unit 730 is constituted by using a card reader that reads the IC card, or the like. Note that, the detection unit 730 may identify users by using user's facial feature point which are set in advance and known pattern matching with respect to an image corresponding to image data generated by imaging faces of the plurality of users, and may output the identification result to the information processing apparatus le. The detection unit 730 may identify users based on signals which are input in accordance with operations from the operating unit 37, and may output the identification result to the information processing apparatus 1e.

Configuration of Information Processing Apparatus

Next, a configuration of the information processing apparatus le will be described.

The information processing apparatus le includes a control unit 32e, a recording unit 34e, and an analysis unit 40e in substitution for the control unit 32d, the recording unit 34d, and the analysis unit 40d of the information processing apparatus 1d according to the fifth embodiment.

The control unit 32e is constituted by using a CPU, an FPGA, a GPU, or the like, and controls the first wearable device 710, the second wearable device 720, the detection unit 730, and the display unit 20. The control unit 32e includes an identification detection controller 327 in addition to the gaze detection controller 321, the voice input controller 322, and the display controller 323.

The identification detection controller 327 control the detection unit 730, identifies each of the plurality of users based on an acquisition result that is acquired by the detection unit 730, and outputs the identification result to the recording unit 34e.

The recording unit 34e is constituted by using a volatile memory, a nonvolatile memory, a recording medium, or the like. The recording unit 34e further includes an identification information recording unit 348 in addition to the configuration of the recording unit 34c according to the fourth embodiment.

The identification information recording unit 348 records pieces of identification information of the plurality of users which are input from the identification detection controller 327.

The analysis unit 40e analyzes the degree of attention of a gaze (gaze point) by detecting any one of a movement speed of the gaze, a movement distance of the gaze in a constant time, and a residence time of the gaze in a constant area based on gaze data that is correlated with the same time axis as in the voice data. In addition, the analysis unit 40e allocates the corresponding gaze period and the textual information converted by the converter 35 to the gaze data based on the degree of attention that is analyzed, the important period of the voice data which is set by the setting unit 38, and the identification information that is recorded by the identification information recording unit 348, and records the corresponding gaze period and the textual information in the recording unit 34e. Specifically, the analysis unit 40e allocates a value, which is obtained by multiplying the degree of attention that is analyzed by a coefficient corresponding to the identification information of each user which is recorded by the identification information recording unit 348 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34e. That is, the analysis unit 40e performs processing so that the more a user is important (for example, a rank set in accordance with a duty), the higher the rank of the corresponding gaze period becomes. The analysis unit 40e is constituted by using a CPU, an FPGA, a GPU, or the like.

Processing of Information Processing System

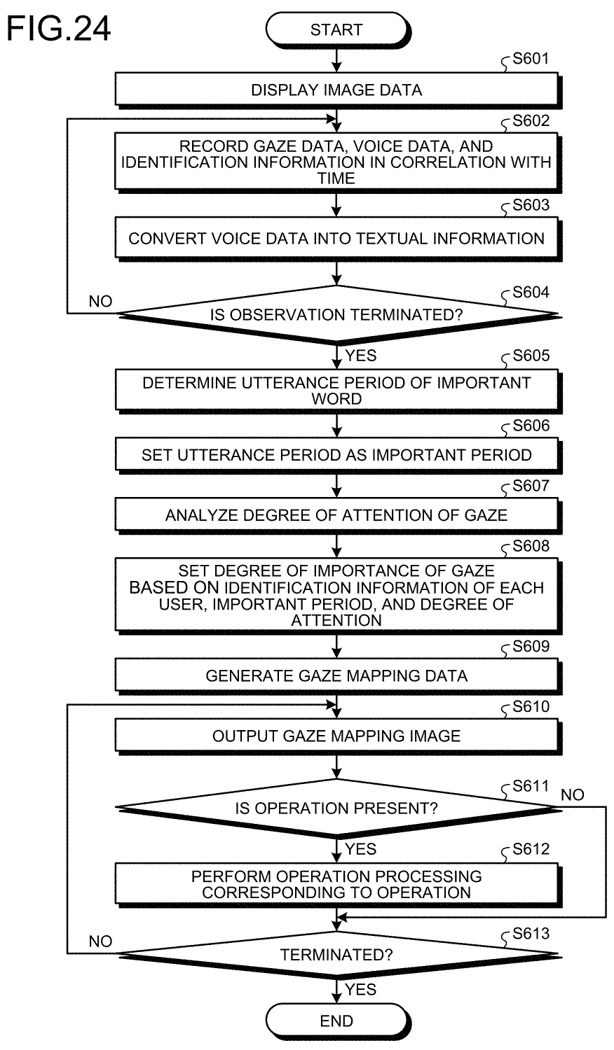

Next, processing that is executed by the information processing system 700 will be described. FIG. 24 is a flowchart illustrating an outline of the processing that is executed by the information processing system 700.

As illustrated in FIG. 24, the display controller 323 causes the display unit 20 to display an image corresponding to the image data that is recorded by the image data recording unit 343 (Step S601).

Next, the control unit 32e records the gaze data that is generated by each of the first wearable device 710 and the second wearable device 720, the voice data, and the identification information that is acquired by the detection unit 730 in the gaze data recording unit 341, the voice data recording unit 342, and the identification information recording unit 348 in correlation with time that is measured by the time measurement unit 33 (Step S602). After Step S602, the information processing system 700 transitions to Step S603.

Step S603 to Step S607 respectively corresponds to Step S303 to Step S307 in FIG. 12. After Step S607, the information processing system 700 transitions to Step S608 to be described later.

Next, the analysis unit 40e allocates a value, which is obtained by multiplying the degree of attention that is analyzed by a coefficient corresponding to the identification information of each user which is recorded by the identification information recording unit 348 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34e (Step S608).

Step S609 to Step S613 respectively corresponds to Step S309 to Step S313 in FIG. 12.

According to the above-described fifth embodiment, the analysis unit 40e allocates a value, which is obtained by multiplying the degree of attention that is analyzed by a coefficient corresponding to the identification information of each user which is recorded by the identification information recording unit 348 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34e. Accordingly, the degree of importance based on the identification information and the degree of attention can be allocated to first voice data or second voice data, and thus it is possible to understand the important period of the voice data in consideration of the degree of attention that corresponds to a user.

Note that, in the sixth embodiment, the analysis unit 40e allocates a value, which is obtained by multiplying the degree of attention that is analyzed by a coefficient corresponding to the identification information of each user which is recorded by the identification information recording unit 348 and a coefficient corresponding to a keyword of the important period that is set by the setting unit 38, to the gaze period (time) of the degree of attention of the gaze data which corresponds to a period before and after the important period of the voice data as the corresponding gaze period, and records the corresponding gaze period in the recording unit 34e, but there no limitation thereto. For example, a position of each of the plurality of users may be detected, and a value, which is obtained by multiplying the detection result by a coefficient corresponding to a keyword of the important period that is set by the setting unit 38, may be allocated to the gaze period (time) of the degree of attention of each of first gaze data and the second gaze data which correspond to a period before and after the important period of the voice data as the corresponding gaze period, and the corresponding gaze period may be recorded in the recording unit 34e.

### Other Embodiments

The present disclosure can be accomplished by appropriately combining a plurality of constituent elements which are disclosed in the first to sixth embodiments. For example, several constituent elements may be removed from all constituent elements which are described in the first to fifth embodiments. In addition, the constituent elements described in the first to sixth embodiments may be appropriately combined.

In addition, in the first to sixth embodiments, the “unit” may be replaced with “means”, “circuit”, or the like. For example, the control unit may be replaced with control means or a control circuit.

In addition, a program that is executed by the information processing apparatuses according to the first to sixth embodiments is provided as file data in a format that can be installed or in a format that can be executed in a state of being recorded on a computer-readable recording medium such as a CD-ROM, a flexible disk (FD), a CD-R, a digital versatile disk (DVD), a USB medium, and a flash memory.

In addition, the program that is executed by the information processing apparatus according to the first to fifth embodiments may be stored in a computer that is connected to a network such as the Internet, and may be downloaded through the network. In addition, the program that is executed by the information processing apparatus according to the first to fifth embodiments may be provided or distributed through a network such as the Internet.

In addition, in the first to fifth embodiments, a signal is transmitted from various devices through a transmission cable. However, for example, it is not necessary for the signal to be transmitted in a wired manner, and the signal may be transmitted in a wireless manner. In this case, the signal may be transmitted from the devices in conformity to a predetermined wireless communication standard (for example, Wi-Fi (registered trademark) or Bluetooth (registered trademark)). Wireless communication may be performed in conformity to another wireless communication standard.

Note that, in descriptions of the flowcharts in this specification, the sequence of processing between steps is stated by using expressions such as “first”, “then”, and “next”, but the sequence of the processing which is necessary to carry out the present disclosure is not uniquely determined by the expressions. That is, the sequence of processing in the flowcharts described in this specification can be changed in a range without contradictions.

According to the present disclosure, an effect capable of understanding a gaze area corresponding to the degree of importance of a voice is attained.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An information processing apparatus comprising:
a processor comprising hardware, the processor being configured to execute:
setting an utterance period, in which an uttering voice includes a keyword having an importance degree of a predetermined value or more, as an important period with respect to user's voice data input from an external device; and
allocating a corresponding gaze period corresponding to the set important period to gaze data that is input from an external device and is correlated with the same time axis as in the voice data, and recording the corresponding gaze period in a memory.

2. The information processing apparatus according to claim 1,
wherein the processor sets the important period based on important word information with which a keyword that is input from the external device and an index are correlated.

3. The information processing apparatus according to claim 1,
wherein the processor sets the important period based on important word information with which each of a plurality of keywords registered in advance and an index are correlated.

4. The information processing apparatus according to claim 1,
wherein the processor extracts a gaze period, for which a degree of attention of a gaze of the user is analyzed, based on the gaze data, and allocates the corresponding gaze period to the gaze period of the gaze data before and after the important period of the voice data based on the gaze period and the important period.

5. The information processing apparatus according to claim 4,
wherein the processor analyzes the degree of attention by detecting any one of a movement speed of the gaze, a movement distance of the gaze in a constant time, and a residence time of the gaze in a constant area.

6. The information processing apparatus according to claim 1, further comprising:
a converter configured to convert the voice data to textual information,
wherein the keyword is a type of the textual information, and
the processor sets the important period based on the textual information and the keyword.

7. The information processing apparatus according to claim 6, wherein
the processor generates gaze mapping data in which the corresponding gaze period and coordinate information of the corresponding gaze period are correlated with an image corresponding to image data that is input from an external device.

8. The information processing apparatus according to claim 7, wherein the processor
analyzes a trajectory of a gaze of the user based on the gaze data, and
correlates the trajectory with the image to generate the gaze mapping data.

9. The information processing apparatus according to claim 7, further comprising:
a display controller configured to control a display to display a gaze mapping image corresponding to the gaze mapping data, and controls the display to highlight at least partial area of the gaze mapping data which corresponds to the corresponding gaze period.

10. The information processing apparatus according to claim 7,
wherein the processor correlates the coordinate information with the textual information to generate the gaze mapping data.

11. The information processing apparatus according to claim 7, further comprising a display controller configured to control a display to display a gaze mapping image corresponding to the gaze mapping data,
wherein the processor extracts a keyword designated in accordance with an operation signal that is input from an external device from the textual information, and
the display controller controls the display to highlight at least partial area of the gaze mapping data that is correlated with the extracted keyword, and controls the display to display the extracted keyword.

12. The information processing apparatus according to claim 1, further comprising:
a gaze detector configured to continuously detect a gaze of the user and generate the gaze data; and
a voice input unit configured to receive an input of voice of the user and generate the voice data.

13. The information processing apparatus according to claim 4, further comprising:
a detector configured to detect identification information for identifying each of a plurality of users,
wherein the processor analyzes the degree of attention of each of the plurality of users based on a plurality of pieces of the gaze data which are obtained by detecting each of lines of sight of the plurality of users, and allocates the corresponding gaze period to the gaze data of each of the plurality of users based on the degree of attention and the identification information.

14. The information processing apparatus according to claim 12, further comprising:
a microscope including an eyepiece portion which is capable of changing an observation magnification set to observe a specimen, and with which the user is capable of observing an observation image of the specimen; and
an imaging sensor connected to the microscope, and configured to capture the observation image of the specimen and generate image data,
wherein the gaze detector is provided in the eyepiece portion of the microscope, and
the processor performs weighting of the corresponding gaze period in accordance with the observation magnification.

15. The information processing apparatus according to claim 12, further comprising:
an endoscope including an imaging sensor provided at a distal end of an insertion portion capable of being inserted into a subject and configured to capture images of an inner side of the subject and generate image data, and an operating unit configured to receive an input of operation for changing a field of view.

16. The information processing apparatus according to claim 15,
wherein the processor performs weighting of the corresponding gaze period based on an operation history related to the input of operation.

17. A method for information processing, the method comprising:
setting an utterance period, in which an uttering voice includes a keyword having an importance degree of a predetermined value or more, as an important period with respect to user's voice data input from an external device; and
allocating a corresponding gaze period corresponding to the set important period to gaze data that is input from an external device and is correlated with the same time axis as in the voice data, and recording the corresponding gaze period in a memory.

18. A non-transitory computer readable recording medium on which an executable program is recorded, the program instructing a processor to execute:
setting an utterance period, in which an uttering voice includes a keyword having an importance degree of a predetermined value or more, as an important period with respect to user's voice data input from an external device; and
allocating a corresponding gaze period corresponding to the set important period to gaze data that is input from an external device and is correlated with the same time axis as in the voice data, and recording the corresponding gaze period in a memory.
